(19) United States
(12) Patent Application Publication
(10) Pub. No.: US 2006/0264957 A1
(43) Pub. Date: Nov. 23, 2006

(54) APPARATUS FOR PERFORMING A DISCECTOMY THROUGH A TRANS-SACRAL AXIAL BORE WITHIN THE VERTEBRAE OF THE SPINE

(75) Inventors: Andrew H. Cragg, Edina, MN (US); Jonathan Kagan, Hopkins, MN (US)

Correspondence Address: KNOBBE MARTENS OLSON & BEAR LLP, 2040 MAIN STREET, FOURTEENTH FLOOR, IRVINE, CA, 92614, US

(73) Assignee: TranS1, Inc.

(21) Appl. No.: 11/460,892

(22) Filed: Jul. 28, 2006

Related U.S. Application Data
(63) Continuation of application No. 09/782,534, filed on Feb. 13, 2001.
(60) Provisional application No. 60/182,748, filed on Feb. 16, 2000.

(51) Int. Cl. A61B 17/00

(52) U.S. Cl. 606/80

(57) ABSTRACT

Methods and apparatus for and performing a partial or complete discectomy of an intervertebral spinal disc accessed by one or more trans-sacral axial spinal instrumentation/fusion (TASIF) axial bore formed through vertebral bodies in general alignment with a visualized, trans-sacral anterior or posterior axial instrumentation/fusion line (AAIFL or PAIFL) line. A discectomy instrument is introduced through the axial bore, the axial disc opening, and into the nucleus to locate a discectomy instrument cutting head at the distal end of the discectomy instrument shaft within the nucleus. The cutting head is operated by operating means coupled to the instrument body proximal end for extending the cutting head laterally away from the disc opening within the nucleus of the intervertebral spinal disc and for operating the cutting head to form a disc cavity within the annulus extending laterally and away from the disc opening or a disc space wherein the disc cavity extends through at least a portion of the annulus. A discectomy sheath that is first introduced to extend from the skin incision through the axial bore and into the axial disc opening having a discectomy sheath lumen that the discectomy instrument is introduced through. The discectomy sheath is preferably employed for irrigation and aspiration of the disc cavity or just aspiration if irrigation fluids are introduced through a discectomy instrument shaft lumen. The cutting head of the discectomy tool is deflected from the sheath lumen laterally and radially toward the annulus using a deflecting catheter or pull wire.

This application is a continuation application of U.S. patent application Ser. No. 09/782,534 filed on Feb. 13, 2001 which claims priority and benefits from Provisional Patent Application No. 60/182,748, filed Feb. 16, 2000, entitled METHOD AND APPARATUS FOR TRANS-SACRAL SPINAL FUSION.

### CROSS-REFERENCE TO RELATED APPLICATIONS

Reference is hereby made to commonly assigned co-pending (1) U.S. patent application Ser. No. 09/640,222 filed Aug. 16, 2000, for METHOD AND APPARATUS FOR PROVIDING POSTERIOR OR ANTERIOR TRANS-SACRAL ACCESS TO SPINAL VERTEBRAE in the name of Andrew H. Cragg, MD; (2) Ser. No. 09/684,620 filed Oct. 10, 2000, for AXIAL SPINAL IMPLANT AND METHOD AND APPARATUS FOR IMPLANTING AN AXIAL SPINAL IMPLANT WITHIN THE VERTEBRAE OF THE SPINE in the name of Andrew H. Cragg, MD; (3) Ser. No. 09/709,105 filed Nov. 10, 2000, for METHODS AND APPARATUS FOR FORMING CURVED AXIAL BORES THROUGH SPINAL VERTEBRAE in the name of Andrew H. Cragg, MD et al.; (4) Ser. No. 09/710,369 filed Nov. 10, 2000, for METHODS AND APPARATUS FOR FORMING SHAPED AXIAL BORES THROUGH SPINAL VERTEBRAE in the name of Andrew H. Cragg, MD et al.; and (5) (9500010.APP) filed Feb. 13, 2001, for METHODS AND APPARATUS FOR PERFORMING THERAPEUTIC PROCEDURES IN THE SPINE in the name of Andrew H. Cragg, MD.

### FIELD OF THE INVENTION

The present invention relates generally to spinal surgery, particularly methods and apparatus for forming one or more trans-sacral axial spinal instrumentation/fusion (TASIF) axial bore through vertebral bodies in general alignment with a visualized, trans-sacral anterior or posterior axial instrumentation/fusion line (AAIFL or PAIFL) line in a minimally invasive, low trauma, manner and performing a partial or complete discectomy of an intervertebral spinal disc through the axial bore.

### BACKGROUND OF THE INVENTION

It has been estimated that 70% of adults have had a significant episode of back pain or chronic back pain emanating from a region of the spinal column or backbone. Many people suffering chronic back pain or an injury requiring immediate intervention resort to surgical intervention to alleviate their pain.

The spinal column or backbone encloses the spinal cord and consists of 33 vertebrae superimposed upon one another in a series which provides a flexible supporting column for the trunk and head. The vertebrae cephalad (i.e., toward the head or superior) to the sacral vertebrae are separated by fibrocartilaginous intervertebral spinal discs and are united by articular capsules and by ligaments. The uppermost seven vertebrae are referred to as the cervical vertebrae, and the next lower twelve vertebrae are referred to as the thoracic, or dorsal, vertebrae. The next lower succeeding five vertebrae below the thoracic vertebrae are referred to as the lumbar vertebrae and are designated L1-L5 in descending order. The next lower succeeding five vertebrae below the lumbar vertebrae are referred to as the sacral vertebrae and are numbered S1-S5 in descending order. The final four vertebrae below the sacral vertebrae are referred to as the coccygeal vertebrae. In adults, the five sacral vertebrae fuse to form a single bone referred to as the sacrum, and the four rudimentary coccyx vertebrae fuse to form another bone called the coccyx or commonly the “tail bone”. The number of vertebrae is sometimes increased by an additional vertebra in one region, and sometimes one may be absent in another region.

Typical lumbar, thoracic and cervical vertebrae consist of a ventral or vertebral body and a dorsal or neural arch. In the thoracic region, the ventral body bears two costal pits for reception of the head of a rib on each side. The arch which encloses the vertebral foramen is formed of two pedicles and two lamina. A pedicle is the bony process which projects backward or anteriorly from the body of a vertebra connecting with the lamina on each side. The pedicle forms the root of the vertebral arch. The vertebral arch bears seven processes: a dorsal spinous process, two lateral transverse processes, and four articular processes (two superior and two inferior). A deep concavity, inferior vertebral notch, on the inferior border of the arch provides a passageway or spinal canal for the delicate spinal cord and nerves. The successive disorders and various treatments that have been clinically used or proposed are first described as follows.

With aging, the nucleus becomes less fluid and more viscous and sometimes even dehydrates and contracts (sometimes referred to as “isolated disc resorption”) causing severe pain in many instances. In addition, the annulus tends to thicken, desiccate, and become more rigid, lessening its ability to elastically deform under load and making it susceptible to fracturing or fissuring.

One form of degeneration of the disc occurs when the annulus fissures or is rent. The fissure may or may not be accompanied by extrusion of nucleus material into and beyond the annulus. The fissure itself may be the sole morphological change, above and beyond generalized degenerative changes in the connective tissue of the disc, and disc fissures can nevertheless be painful and debilitating. Biochemicals contained within the nucleus are alleged to escape through the fissure and irritate nearby structures.

A fissure also may be associated with a herniation or rupture of the annulus causing the nucleus to bulge outward or extrude out through the fissure and impinge upon the spinal column or nerves (a “ruptured” or “slipped” disc). With a contained disc herniation, the nucleus may work its way partly through the annulus but is still contained within the annulus or beneath the posterior longitudinal ligament, and there are no free nucleus fragments in the spinal canal. Nevertheless, even a contained disc herniation is problematic because the outward protrusion can press on the spinal cord or on spinal nerves causing sciatica.

Another disc problem occurs when the disc bulges outward circumferentially in all directions and not just in one location. This occurs when over time, the disc weakens, bulges outward and takes on a “roll” shape. Mechanical stiffness of the joint is reduced and the spinal motion segment may become unstable shortening the spinal cord segment. As the disc “roll” extends beyond the normal circumference, the disc height may be compromised, and foramina with nerve roots are compressed causing pain. In addition, osteophytes may form on the outer surface of the disc roll and further encroach on the spinal canal and foramina through which nerves pass. The cephalad vertebra may eventually settle on top of the caudal vertebra. This condition is called “lumbar spondylosis”.

In addition, various types of spinal column displacement disorders are known in one or more spinal motion segment that are hereditary or are caused by degenerative disease processes or trauma. Such spinal displacement disorders include scoliosis (abnormal lateral curvature of the spine), kyphosis (abnormal forward curvature of the spine, usually in the thoracic spine), excess lordosis (abnormal backward curvature of the spine, usually in the lumbar spine), spondylolisthesis (forward displacement of one vertebra over another, usually in the lumbar or cervical spine). At times, the displacement disorder is accompanied by or caused by a fracture or partial collapse of one or more vertebrae or degeneration of a disc. Patients who suffer from such conditions can experience moderate to severe distortion of the thoracic skeletal structure, diminished ability to bear loads, loss of mobility, extreme and debilitating pain, and oftentimes suffer neurologic deficit in nerve function.

Approximately 95% of spinal surgery involves the lower lumbar vertebrae designated as the fourth lumbar vertebra (“L4”), the fifth lumbar vertebra (“L5”), and the first sacral vertebra (“S1”). Persistent low back pain is attributed primarily to degeneration of the spinal disc connecting L5 and S1. Traditional, conservative methods of treatment include bed rest, pain and muscle relaxant medication, physical therapy or steroid injection. Upon failure of conservative therapy spinal pain (assumed to be due to instability) has traditionally been treated by spinal fusion, with or without instrumentation, which causes the vertebrae above and below the disc to grow solidly together and form a single, solid piece of bone.

Highly invasive, open surgical procedures have been developed and used to perform a “complete discectomy” to surgically remove the disc, and the vertebral bodies are then fused together. The removal of the disc involves removing the nucleus, cutting away the cartilaginous endplates adhered to the opposed cortical bone endplates of the cephalad and caudal vertebral bodies, and removing at least a portion of the annulus. Fusion of the vertebral bodies involves preparation of the exposed endplate surfaces by decortication (scraping the endplate cortical bone) and the deposition of additional bone into disc space between the prepared endplate surfaces. The complete discectomy and fusion may be performed through a posterior surgical route (from the back side of the patient) or an anterior surgical route (from the front side of the patient). The removed vertebral bone may be just the hard cortical bone or may include soft cancellous soft bone in the interior of the vertebral bodies. Controversy exists regarding the preferred method of performing these fusions for various conditions of the spine. Sometimes, non-biological materials are used to augment and support the bone grail (fixation systems). Sometimes, the fixation is performed from the posterior route (posterior fixation), or from the anterior route (anterior fixation), or even both sides (anterior-posterior fixations or circumferential fusion).

Current treatment methods other than spinal fusion for symptomatic disc rolls and herniated discs include “laminectomy” which involves the lateral surgical exposure of the annulus and surgical excision of the symptomatic portion of the herniated disc followed by a relatively lengthy recuperation period.

Various other surgical treatments that attempt to preserve the intervertebral spinal disc and to simply relieve pain include a “nucleotomy” or “disc decompression” to remove some or most of the interior nucleus thereby decompressing and decreasing outward pressure on the annulus. In less invasive microsurgical procedures known as “microlumbar discectomy” and “automated percutaneous lumbar discectomy”, the nucleus is removed by suction through a needle laterally extended through the annulus. Although these procedures are less invasive than open surgery, they nevertheless suffer the possibility of injury to the nerve root and dural sac, perineural scar formation, reherniation of the site of the surgery, and instability due to excess bone removal. Moreover, they involve the perforation of the annulus.

Another method of treatment is known as “chemonucleolysis”, which is carried out by injection of the enzyme chymopapain into the nucleus through the annulus. This procedure has many complications including severe pain and spasm, which may last up to several weeks following injection. Sensitivity reactions and anaphylactic shock occur in limited but significant numbers of patients.

Although damaged discs and vertebral bodies can be identified with sophisticated diagnostic imaging, the surgical procedures are so extensive that clinical outcomes are not consistently satisfactory. Furthermore, patients undergoing such fusion surgery experience significant complications and uncomfortable, prolonged convalescence. Surgical complications include disc space infection, nerve root injury, hematoma formation, and instability of adjacent vertebrae.

Many surgical techniques, instruments and spinal disc implants have been described in the medical literature and in patents that are directed to providing less invasive, percutaneous, lateral access to a degenerated intervertebral spinal disc. Then, instruments are introduced through lateral disc openings made through the annulus for performing a discectomy and implanting bone growth materials or biomaterials or spinal disc implants inside the annulus. Or, one or more laterally extending space or hole is bored through the disc to receive one or more laterally inserted spinal disc implant or bone growth material to promote fusion or to receive a preformed, artificial, functional disc replacement implant as typified by U.S. Pat. No. 5,700,291.

Percutaneous lateral procedures and instruments for performing such discectomies are disclosed in U.S. Pat. Nos. Re. 33,258, 4,573,448, 5,015,255, 5,313,962, 5,383,884, 5,702,454, 5,762,629, 5,976,146, 6,095,149, and 6,127,597 and in PCT publication WO 99/47055, for example. A laparascopic technique and apparatus for traversing the retroperitoneal space from an abdominal skin incision to an anterior surface of the disc annulus and performing a discoscopy is disclosed in the '962 patent, for example. Percutaneous surgical disc procedures and apparatus that accesses the disc in a posterolateral approach from a skin incision in the patient's back are described in the '629 and '448 patents, for example.

The nucleus is fragmented by various mechanical cutting heads as disclosed, for example in the '258, '962, '884, and '597 patents, for example. Or, thermal or laser energy is applied to desiccate the nucleus and to stiffen the annulus as described in the '149 patent, for example. Or, the nucleus and portions of the cephalad and caudal vertebral bodies are mechanically cut away to enlarge the disc space as described in the PCT '055 publication and in the '255 patent, for example. Irrigation fluid is introduced into the disc space or cavity and the fragments or desiccation by-products of the nucleus and any bone and annulus fragments are aspirated from the disc space or cavity. The irrigation and aspiration is effected through an access cannula positioned against the opening through the annulus of the herniated disc as disclosed in the '629 patent, for example, or through a lumen of the discectomy instrument, as disclosed in the '258 patent, for example. A measure of safety and accuracy is added to these operative procedures by the artiroscopic visualization of the annulus and other important structures which lie in the path of the instruments, such as the spinal nerve.

The above-described procedures involve invasive surgery that laterally exposes the anterior or posterior (or both) portions of the vertebrae and intervertebral spinal disc. Extensive muscular stripping and bone preparation can be necessary. As a result, the spinal column can be further weakened and/or result in surgery induced pain syndromes. Thus, presently used or proposed surgical fixation and fusion techniques involving the lower lumbar vertebrae suffer from numerous disadvantages.

Methods and apparatus for accessing the discs and vertebrae by lateral surgical approaches that purportedly reduce muscular stripping (and that are similar to those disclosed in the above-referenced '629 and '888 patents) are described in U.S. Pat. No. 5,976,146. The intervening muscle groups or other tissues are spread apart by a cavity forming and securing tool set disclosed in the '146 patent to enable endoscope aided, lateral access to damaged vertebrae and discs and to perform corrective surgical procedures. However, it is preferable to avoid the lateral exposure to correct less severe spondylolisthesis and other spinal injuries or defects affecting the lumbar and sacral vertebrae and discs.

A less intrusive posterior approach for treating spondylolisthesis is disclosed in U.S. Pat. No. 6,086,589, wherein a straight bore is formed through the sacrum from the exposed posterior sacral surface and in a slightly cephalad direction into the L5 vertebral body, preferably after realigning the vertebrae. A straight, hollow, threaded shaft with side wall holes restricted to the end portions thereof and bone growth material are inserted into the bore. A discectomy of the disc between L5 and S1 is preferably performed in an unexplained manner, and bone ingrowth material is also preferably inserted into the space between the cephalad and caudal vertebral bodies. Only a limited access to and alignment of S1 and L5 can be achieved by this approach because the distal ends of the straight bore and shaft approach and threaten to perforate the anterior surface of the L5 vertebral body. This approach is essentially a posteriolateral approach that is intended to fuse S1 and L5 and cannot access more cephalad vertebral bodies or intervertebral spinal discs.

Drilling tools are employed in many of the above described surgical procedures to bore straight holes into the vertebral bones. The boring of curved bores in other bones is described in U.S. Pat. Nos. 4,265,231, 4,541,423, and 5,002,546, for example. The '231 patent describes an elongated drill drive shaft enclosed within a pre-curved outer sheath that is employed to drill curved suture holding open ended bores into bones so that the suture passes through both open ends of the bore. The '423 patent describes an elongated flexible drill drive shaft enclosed within a malleable outer sheath that can be manually shaped into a curve before the bore is formed. The '546 patent describes a complex curve drilling tool employing a pivotal rocker arm and curved guide for a drill bit for drilling a fixed curve path through bone. All of these approaches dictate that the curved bore that is formed follow the predetermined and fixed curvature of the outer sheath or guide. The sheath or guide is advanced through the bore as the bore is made, making it not possible for the user to adjust the curvature of the bore to track physiologic features of the bone that it traverses.

All of the above-described patents and other patents referenced herein that access a single spinal disc to perform a discectomy, do so from a lateral approach that involves weakening of the spinal fusion segment. There remains a need for methods and apparatus for performing a discectomy of an intervertebral spinal disc in a minimally invasive, low trauma, manner.

### SUMMARY OF THE INVENTION

The preferred embodiments of the invention involve methods and apparatus for performing a discectomy of one or more spinal disc in the human spine having an anterior aspect, a posterior aspect and an axial aspect, in a minimally invasive, low trauma, manner.

The discectomy apparatus of the present invention is operable through a trans-sacral axial bore extending cephalad and axially from a sacral position of a sacral vertebral body through one or more vertebral body and through an axial disc opening into the nucleus of the intervertebral spinal disc. The discectomy apparatus comprises a discectomy instrument that is introduced through the axial bore, the axial disc opening and into the nucleus to locate a discectomy instrument cutting head at the distal end of the discectomy instrument shaft within the nucleus. The cutting head is operated by operating means coupled to the instrument body proximal end for extending the cutting head laterally away from the disc opening within the nucleus of the intervertebral spinal disc and for operating the cutting head to form a disc cavity within the annulus extending laterally and away from the disc opening or a disc space wherein the disc cavity extends through at least a portion of the annulus.

The discectomy apparatus optionally comprises a discectomy sheath that is first introduced to extend from the skin incision through the axial bore and into the axial disc opening having a discectomy sheath lumen that the discectomy instrument is introduced through. The discectomy instrument is dimensioned to fit within and to extend through the sheath lumen to enable extension of the cutting head from the sheath lumen distal end opening. The discectomy sheath is preferably employed for irrigation and aspiration of the disc cavity or just aspiration if irrigation fluids are introduced through a discectomy instrument shaft lumen.

The accessing of the posterior sacral position is preferably performed by surgically exposing the posterior target point. One or more posterior TASIF axial bore is formed starting from the exposed posterior target point and extending axially (that is in the axial aspect of the spinal column) in the cephalad direction in alignment with the visualized, trans-sacral PAIFL. The posterior TASIF axial bore(s) has a curvature aligned with the anatomical curvature of the sacral and lumbar vertebrae cephalad to the posterior target point so that the posterior trans-sacral axial bore(s) can extend in the cephalad direction to a cephalad bore end in one of the lumbar vertebral bodies or discs.

Preferably, an anterior access tract is formed extending from a skin incision through presacral space to the anterior target point. One or more anterior TASIF axial bore is formed starting from the accessed anterior target point and extending axially in the cephalad direction in alignment with the visualized trans-sacral AAIFL. The anterior TASIF axial bore(s) is either straight or is curved to follow the anatomical curvature of the sacral and lumbar vertebrae cephalad to the accessed anterior target point and extend in the cephalad direction to a cephalad bore end in one of the lumbar vertebral bodies or discs.

In either case, the “alignment” of a single anterior or posterior TASIF axial bore is either co-axial or parallel alignment with the visualized AAIFL or PAIFL, respectively. The alignment of a plurality of anterior or posterior TASIF axial bores is either parallel or diverging alignment with the visualized AAIFL or PAIFL, respectively. All such alignments are defined herein as axial.

Then, a partial discectomy or a complete discectomy of a spinal disc accessed through the TASIF axial bore is performed employing discectomy apparatus of the present invention. In the context of the present invention, a “partial discectomy” involves removal or desiccation of any portion of the nucleus through the axial disc opening, whereas a “complete discectomy” involves perforation or removal of at least some of the annulus of the intervertebral spinal disc.

The cutting head at the distal end of the discectomy instrument comprises one of a mechanical cutting tool for fragmenting and/or an energy emitter for desiccating a portion of the nucleus in a partial discectomy to form a disc cavity within the annulus or both the nucleus and at least a portion of the annulus in a complete discectomy. The cutting tools can be vibrated by ultrasonic energy vibrations transmitted from the discectomy instrument shaft proximal end through the shaft body and to the cutting head. A further type of cutting tool can comprise a water jet that applies high pressure water bursts against the nucleus or annulus to fragment the tissue. The emitted energy directly or indirectly heats adjacent tissue, and the desiccation of the annulus and/or nucleus is effected as by localized shrinking or burning or drying accompanied by applied mechanical force in certain cases.

A first preferred type of discectomy instrument comprises a cutting head that is mounted to the distal end of a flexible discectomy instrument shaft that can traverse the short radius bend at the axial disc opening laterally into the annulus. The cutting head is axially aligned with the discectomy instrument shaft when unrestrained and when advanced through the TASIF axial bore or discectomy sheath lumen. But, a bend can be formed in the discectomy instrument shaft proximal to the cutting head to allow the deflection of the cutting head laterally and radially away from the axial disc opening. Then, as the cutting head is extended further laterally toward the annulus, the discectomy instrument shaft bends as it passes through the axial disc opening.

With certain cutting heads employed in this type, the cutting head is swept through the annulus and/or nucleus in one or more prescribed arc as the cutting head is advanced toward or through the annulus to form an irregular disc cavity or disc space. Or the cutting head is rotated through 360° as the cutting head is advanced to a prescribed radius to form a generally circular disc cavity within the annulus.

In one variation of the first preferred type, the distal end of the discectomy instrument shaft may be angularly deflected using a deflection mechanism, e.g., a pull wire within a pull wire lumen of the elongated, flexible, discectomy instrument shaft and a proximal pull wire control of a proximal guiding and cutting mechanism coupled thereto. The pull wire is released during advancement of the cutting head through the discectomy sheath lumen or directly through the TASIF axial bore and into the axial disc opening. Then, the pull wire is retracted to form a bend to at least initially direct the cutting head laterally and radially away from the axial disc opening. Further lateral extension of the cutting head toward the annulus is obtained by releasing the pull wire and advancing the discectomy instrument shaft distally while the flexible discectomy instrument shaft bends at the axial disc opening.

In a second variation of this first type of discectomy instrument, the deflection of the cutting head with respect to the discectomy instrument shaft is enabled by a deflection catheter having a deflection catheter lumen extending between a deflection catheter proximal and a deflection catheter distal end. A distal portion of the deflection catheter is angled with respect to a proximal portion of the deflection catheter to orient the deflection catheter lumen distal end opening at about 90° with respect to the deflection catheter lumen in the proximal portion of the deflection catheter.

In use, the deflection catheter is extended either through the discectomy sheath lumen within the TASIF axial bore or directly through the TASIF axial bore and into the axial disc opening to locate the distal portion within the nucleus. The advancement of the discectomy instrument shaft proximal end into the deflection catheter lumen causes the cutting head to be deflected laterally and radially away from the deflection catheter lumen distal end opening into the nucleus and toward or through the annulus.

Energy emitting cutting heads fixed to the discectomy instrument shaft distal end include optical laser light emitters, resistance heating elements or electrocautery elements or the like that are energized via an external energy source coupled to proximal ends of conductors extending through the shaft. Again, the discectomy instrument shaft or deflection catheter is rotated to sweep the energy emitter through the nucleus in selected arcs or in full rotation to desiccate the nucleus as the shaft is advanced from the shaft proximal end to advance the energy emitter laterally toward or through the annulus.

Mechanical cutting tools usable in either variation of the first type include flexible cutting wires having fixed ends fixed to the discectomy instrument shaft and free ends optionally having weights at the free ends. The discectomy instrument shaft or the deflection catheter is rotated to sweep the cutting wire through the nucleus to fragment it as the discectomy instrument shaft is advanced from the shaft proximal end to advance the cutting wire laterally toward or through the annulus.

Further types of mechanical cutting tools can also be employed in both variations of the first type, wherein the cutting head is movable with respect to the discectomy instrument shaft to apply force against the disc annulus or nucleus to cut, macerate or fragment tissue. The discectomy instrument comprises a discectomy instrument shaft lumen extending between discectomy instrument shaft proximal and distal ends of the discectomy instrument shaft, a rotatable or outwardly extendable cutting element, and a mechanism extending through the shaft lumen to the cutting element for rotating it or for deploying it. In one variation, a cutting head drive shaft extends through the shaft lumen to a rotatable cutting element, e.g., an augur or drill bit that can be fully exposed or is partially shielded, enabling the rotation of the cutting element by a drive motor coupled with the proximal end of the cutting head drive shaft. In another variation, an extension wire within a discectomy instrument shaft lumen can be advanced distally from the proximal end to advance one or more cutting wire loop bowing outward from one or more side opening of the discectomy instrument shaft adjacent to the shaft distal end to fragment the annulus as the discectomy instrument shaft and cutting wire are rotated. In a further variation, a retraction wire within the discectomy instrument shaft lumen can be retracted proximally from the proximal end to pull back on and bow outward one or more cutting wire loop extending distally from the discectomy instrument shaft distal end to fragment the annulus as the discectomy instrument shaft and cutting wire are rotated.

A still further cutting head usable with the deflection catheter comprises an elongated, flexible, desiccating wire that assumes a planar spiral when unrestrained but is capable of being straightened when inserted through the discectomy sheath lumen or the deflection catheter lumen. The desiccating wire assumes the planar spiral shape with spiral turns pressing outward against the nucleus and toward the annulus as the desiccating wire is extended out of the distal lumen end opening. If the deflection catheter is used, it can be rotated at the proximal shaft end as the spiral shape is formed, and the applied force of the spiral shape causes the nucleus to compress or separate as the spiral forms. Ultrasonic energy can be applied to the desiccating wire as it expands outward and forms the spiral shape, the ultrasonic energy causing the expanding spiral wire shape to vibrate and separate the nucleus or annulus that its turns press against. In still another variation, the spiral shaped desiccating wire can comprise a resistance heating element that is energized to delivery thermal energy to the nucleus as the spiral shape forms and expands.

Yet another type of cutting head usable in either variation of the first type comprises a fluid jet discectomy instrument that has a deflectable tip that is deflected by a pull wire or by the deflection catheter to aim a high pressure fluid jet at a particular direction from the axial disc opening. The fluid jet discectomy instrument shaft is formed with a fluid lumen for conducting pressurized fluid from the discectomy instrument shaft proximal end and out of the distal fluid jet or jets. The discectomy instrument shaft can be advanced and rotated to locate the water jet(s) within prescribed areas of the nucleus and annulus to lyse it into fragments. The emitted fluid and lysed fragments are preferably aspirated through a discectomy sheath lumen that the discectomy instrument shaft is advanced through.

A second preferred type of discectomy instrument comprises a cutting head that extends laterally at about 90° or less outward from the discectomy instrument shaft when the cutting head is released but can be confined within the discectomy sheath lumen or TASIF axial bore during introduction through the TASIF axial bore. Such mechanical cutting heads include stiff brush filaments, single cutting wires or multiple cutting wires or hinged cutting blades which can also have weighted ends.

These cutting tools of the second preferred type can be rotated by a drive motor to form a constant diameter disc cavity that is either contained within the annulus or intrudes into a segment of the disc annulus, depending upon the location of the axial disc opening and the extended radius of the cutting head when fully released into the nucleus.

Thus, in these embodiments and variations, a circular or irregular shaped disc cavity can be formed within the annulus via the axial disc opening, the disc cavity extending laterally and away from the disc opening. In addition, at least portions of the annulus can be removed via the axial disc opening. Certain of the cutting heads can also be manipulated axially to remove to remove at least a portion of the caudal and cephalad cartilaginous endplates and a portion of the caudal and cephalad vertebral body end plates to form an open disc space or disc cavity extending axially into cancellous bone of the caudal and cephalad vertebral bodies.

More than one of the discectomy instruments of the present invention can be employed in the same procedure to remove various features of an intervertebral spinal disc, including herniated or outwardly bulging portions thereof.

More than one TASIF axial bore and axial disc opening to a single spinal disc can be formed, and the discectomy performed through each such TASIF axial bore and axial disc opening.

Other therapeutic procedures including disc augmentation by implantation of disc prosthesis and vertebral fusion by implantation of bone growth encouraging materials or implants can be conducted in the disc cavity or disc space.

When the discectomy or further procedure is completed, the TASIF axial bore(s) is preferably filled to seal the vertebral body(s) and disc(s), to retain any implanted devices and materials in place and/or to align, to fuse and/or to reinforce a fusion zone or the spinal motion segment. The TASIF axial bores can be filled completely or plugged in a section thereof with bone growth materials or pre-formed axial spinal implants or plugs that engage vertebral bone.

This summary of the invention and the objects, advantages and features thereof have been presented here simply to point out some of the ways that the invention overcomes difficulties presented in the prior art and to distinguish the invention from the prior art and is not intended to operate in any manner as a limitation on the interpretation of claims that are presented initially in the patent application and that are ultimately granted.

### BRIEF DESCRIPTION OF THE DRAWINGS

These and other advantages and features of the present invention will be more readily understood from the following detailed description of the preferred embodiments thereof, when considered in conjunction with the drawings, in which like reference numerals indicate identical structures throughout the several views, and wherein:

FIGS. 1-3 are lateral, posterior and anterior views of the lumbar and sacral portion of the spinal column depicting the visualized PAIFL and AAIFL extending cephalad and axially from the posterior laminectomy site and the anterior target point, respectively;

FIG. 4 is a sagittal caudal view of lumbar vertebrae depicting a TASIF axial spinal implant or rod within a TASIF axial bore formed following the visualized PAIFL or AAIFL of FIGS. 1-3;

FIG. 5 is a sagittal caudal view of lumbar vertebrae depicting a plurality, e.g., 2, TASIF axial spinal implants or rods within a like plurality of TASIF axial bores formed in parallel with the visualized PAIFL or AAIFL of FIGS. 1-3;

FIG. 6 is a simplified flow chart showing the principal surgical preparation steps of percutaneously accessing a posterior or anterior target point of the sacrum and forming a percutaneous tract following the visualized PAIFL or AAIFL of FIGS. 1-3, as well as subsequent steps of forming the TASIF bore(s) for treatment of accessed vertebral bodies and intervening discs and of implanting axial spinal implants therein;

FIG. 7 illustrates, in a partial cross-section side view, one manner of obtaining access to a posterior target point for forming a posterior TASIF axial bore through sacral and lumbar vertebrae and intervening discs axially aligned with the visualized PAIFL of FIGS. 1 and 2;

FIG. 8 is an enlarged partial cross section view illustrating a posterior TASIF axial bore through sacral and lumbar vertebrae and intervening discs axially aligned with the visualized PAIFL of FIGS. 1 and 2;

FIG. 9 illustrates, in a partial cross-section side view, one manner of obtaining access to an anterior target point for forming an anterior TASIF axial bore through sacral and lumbar vertebrae and intervening discs axially aligned with the visualized AAIFL of FIGS. 1 and 2;

FIG. 10 is an enlarged partial cross-section view illustrating an anterior TASIF axial bore through sacral and lumbar vertebrae and intervening discs axially aligned with the visualized AAIFL of FIGS. 1 and 2;

FIG. 11 depicts, in a partial cross-section side view, the formation of a plurality of curved TASIF axial bores that diverge apart from a common caudal section in the cephalad direction;

FIGS. 12 and 13 depict, in partial cross-section end views taken along lines 12-12 and 13-13, respectively, of FIG. 7, the divergence of the plurality of curved TASIF axial bores;

FIGS. 14-16 illustrate exemplary shapes of spinal disc cavities and disc spaces that can be formed through TASIF axial bores and axial disc openings employing discectomy instruments of the present invention;

FIG. 17 illustrates, in a partial cross-section side view, a first type of discectomy instrument used to perform a discectomy of a spinal disc through a TASIF axial bore and axial disc opening;

FIG. 18 illustrates, in a partial cross-section side view, a variation of the first type of discectomy instrument used to perform a discectomy of a spinal disc through a TASIF axial bore and axial disc opening;

FIGS. 19-21 illustrate a further embodiment of the first type of discectomy instrument employing a discectomy instrument sheath and deflection catheter for applying laser energy to a spinal disc;

FIGS. 22-24 illustrate a further embodiment of the first type of discectomy instrument employing a discectomy instrument sheath and deflection catheter for applying thermal energy to a spinal disc;

FIGS. 25-27 illustrate a further embodiment of the first type of discectomy instrument employing a discectomy instrument sheath and deflection catheter for applying electrocautery energy to a spinal disc;

FIGS. 28-30 illustrate a further embodiment of the first type of discectomy instrument employing a discectomy instrument sheath and deflection catheter for applying mechanical energy to a spinal disc;

FIGS. 31-33 illustrate a further embodiment of the first type of discectomy instrument employing a discectomy instrument sheath and deflection catheter for applying mechanical energy to a spinal disc;

FIGS. 34-36 illustrate a further embodiment of the first type of discectomy instrument employing a discectomy instrument sheath and deflection catheter for applying mechanical energy to a spinal disc;

FIGS. 37-40 illustrate a further embodiment of the first type of discectomy instrument employing a discectomy instrument sheath and deflection catheter for applying mechanical energy to a spinal disc;

FIGS. 41-43 illustrate a further embodiment of the first type of discectomy instrument employing a discectomy instrument sheath and deflection catheter for applying mechanical energy to a spinal disc;

FIGS. 44-46 illustrate a further embodiment of the first type of discectomy instrument employing a discectomy instrument sheath and deflection catheter for applying mechanical energy to a spinal disc;

FIGS. 47-49 illustrate a first embodiment of the second type of discectomy instrument employing a discectomy instrument sheath for applying mechanical energy to a spinal disc;

FIGS. 50-52 illustrate a further embodiment of the second type of discectomy instrument employing a discectomy instrument sheath for applying mechanical energy to a spinal disc;

FIGS. 53-55 illustrate a further embodiment of the second type of discectomy instrument employing a discectomy instrument sheath for applying mechanical energy to a spinal disc;

FIGS. 56-58 illustrate a further embodiment of the second type of discectomy instrument employing a discectomy instrument sheath for applying mechanical energy to a spinal disc; and

FIGS. 59-61 illustrate a further embodiment of the second type of discectomy instrument employing a discectomy instrument sheath for applying mechanical energy to a spinal disc.

### DETAILED DESCRIPTION OF THE PREFERRED EMBODIMENTS OF THE INVENTION

The methods and surgical instrumentation and axial spinal implants disclosed in the above-referenced provisional application No. 60/182,748 and in the above-referenced co-pending, commonly assigned, related patent applications can be employed in the practice of the present invention.

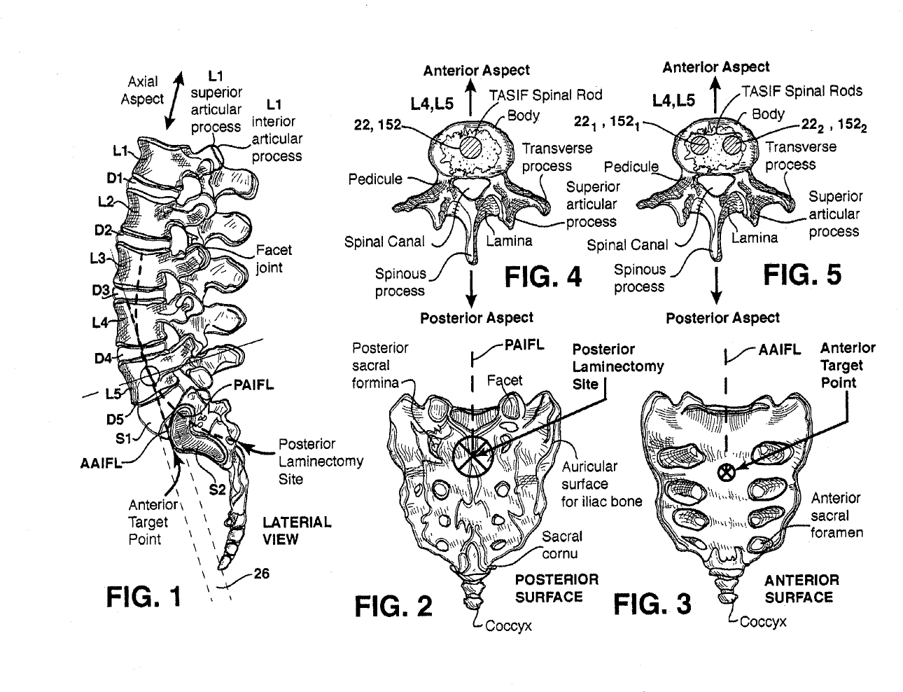

Attention is first directed to the following description of FIGS. 1-6 is taken from the above-referenced parent provisional application No. 60/182,748. The acronyms TASF, AAFL, and PAFL used in the '748 application are changed to TASIF, AAIFL and PAIFL in this application to explicitly acknowledge that instruments can be introduced for inspection or treatments in addition to the fusion and fixation provided by axial spinal implants that may be inserted into the axial bores or pilot holes.

FIGS. 1-3 schematically illustrate the anterior and posterior TASIF surgical approaches in relation to the lumbar region of the spinal column, and FIGS. 4-5 illustrate the location of the TASIF implant or pair of TASIF implants within a corresponding posterior TASIF axial bore 22 or anterior TASIF axial bore 152 or pair of TASIF axial bores 22₁, 22₂ or 152₁, 152₂. Two TASIF axial bores and axial spinal implants or rods are shown in FIG. 5 to illustrate that a plurality, that is two or more, of the same may be formed and/or employed in side by side relation in parallel alignment with the AAIFL or PAIFL or diverging from the AAIFL or PAIFL in the cephalad direction. Preferred TASIF surgical approaches for providing anterior and posterior trans-sacral access depicted in FIGS. 1-3 and preparing the TASIF axial bores 22 or 152 or 22₁, 22₂, or 152₁, 152₂ shown in FIGS. 4 and 5 are illustrated in the above-referenced '105 and '748 applications.

The lower regions of the spinal column comprising the coccyx, fused sacral vertebrae S1-S5 forming the sacrum, and the lumbar vertebrae L1-L5 described above are depicted in a lateral view in FIG. 1. The series of adjacent vertebrae located within the human lumbar and sacral spine have an anterior aspect, a posterior aspect and an axial aspect, and the lumbar vertebrae are separated by intact or damaged intervertebral spinal discs labeled D1-D5 in FIG. 1. FIGS. 2 and 3 depict the posterior and anterior views of the sacrum and coccyx.

The method and apparatus for forming an anterior or posterior TASIF axial bore initially involves accessing an anterior sacral position, e.g. an anterior target point at the junction of S1 and S2 depicted in FIGS. 1 and 3, or a posterior sacral position, e.g. a posterior laminectomy site of S2 depicted in FIGS. 1 and 2. One (or more) visualized, imaginary, axial instrumentation/fusion line extends cephalad and axially in the axial aspect through the series of adjacent vertebral bodies, L4 and L5 in this illustrated example. The visualized AAIFL through L4, D4, L5 and D5 extends relatively straight from the anterior target point along S1 depicted in FIGS. 1 and 3, but may be curved as to follow the curvature of the spinal column in the cephalad direction. The visualized PAIFL extends in the cephalad direction with more pronounced curvature from the posterior laminectomy site of S2 depicted in FIGS. 1 and 2. A preoperative CT scan or magnetic resonance imaging (MRI) study of the patient's spine is conducted to visualize and map the MIFL or PAIFL.

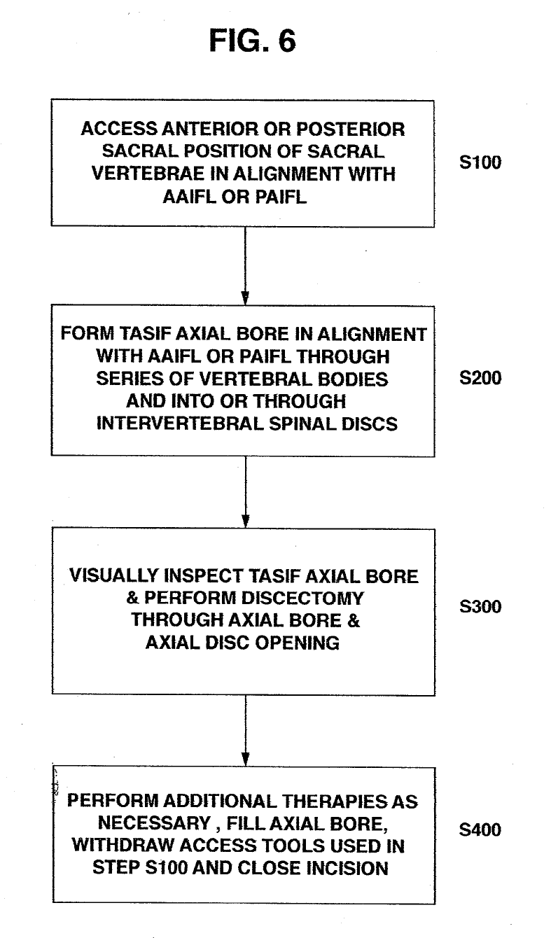

FIG. 6 depicts, in general terms, the surgical steps of accessing the anterior or posterior sacral positions illustrated in FIGS. 1-3 (S100) forming one or more posterior or anterior TASIF axial bore (S200), optionally inspecting the discs and vertebral bodies, performing a discectomy (S300) and optionally performing a further ancillary procedure followed by closing or sealing the axial bore(s) (S400) in a simplified manner. In step S100, access to the anterior or posterior sacral position, that is the anterior target point of FIG. 3 or the posterior laminectomy site of FIG. 2 is obtained, and the anterior or posterior sacral position is penetrated to provide a starting point for each axial bore that is to be created. Then, one or more axial bore is bored from each point of penetration extending in alignment with either the PAIFL or MIFL cephalad and axially through the vertebral bodies of the series of adjacent vertebrae and any intervertebral spinal discs (S200). The axial bore(s) can traverse one or more vertebral body cephalad to the sacral vertebral bodies S1, S2 and any intervertebral disc and can terminate at a cephalad end within a particular vertebral body or spinal disc. The axial bore may be visually inspected using an endoscope to determine if and how the discectomy of step S300 and any ancillary procedure of step S400 should be performed.

Step S100 preferably involves creation of an anterior or posterior percutaneous pathway that enables introduction of further tools and instruments for forming an anterior or posterior percutaneous tract extending from the skin incision to the respective anterior or posterior target point of the sacral surface or, in some embodiments, the cephalad end of a pilot hole over which or through which further instruments are introduced as described in the above-referenced '222 application. The performance of step S100 in the anterior and/or posterior TASIF procedures may involve drilling a pilot hole, smaller in diameter than the TASIF axial bore, in the prescribed alignment with the AAIFL and/or PAIFL in order to complete the formation of the anterior and/or posterior percutaneous tracts.

An “anterior, presacral, percutaneous tract” 26 (FIG. 1) extends through the “presacral space” anterior to the sacrum. The posterior percutaneous tract or the anterior, presacral, percutaneous tract is preferably used to bore one or more respective posterior or anterior TASIF bore in the cephalad direction through one or more lumbar vertebral bodies and intervening discs, if present. “Percutaneous” in this context simply means through the skin and to the posterior or anterior target point, as in transcutaneous or transdermal, without implying any particular procedure from other medical arts. The percutaneous pathway is generally axially aligned with the AAIFL or the PAIFL extending from the respective anterior or posterior target point through at least one sacral vertebral body and one or more lumbar vertebral body in the cephalad direction as visualized by radiographic or fluoroscopic equipment.

It should be noted that the formation of the anterior tract 26 shown in FIG. 1 through presacral space under visualization described above is clinically feasible as evidenced by clinical techniques described by J. J. Trambert, MD, in “Percutaneous Interventions in the Presacral Space: CT-guided Precoccygeal Approach—Early Experience (Radiology 1999; 213:901-904).

The bore forming tool sets comprise elongated drill shaft assemblies supporting distal boring tools, e.g., mechanical rotating drill bits, burrs, augurs, abraders, or the like (collectively referred to as boring heads or drill bits for convenience), that can be manipulated in use to bore a straight or curved axial bore. Suitable bore forming tools are disclosed in the above-referenced provisional application No. 60/182,748 and the '105 application.

Posterior TASIF Axial Bore Formation:

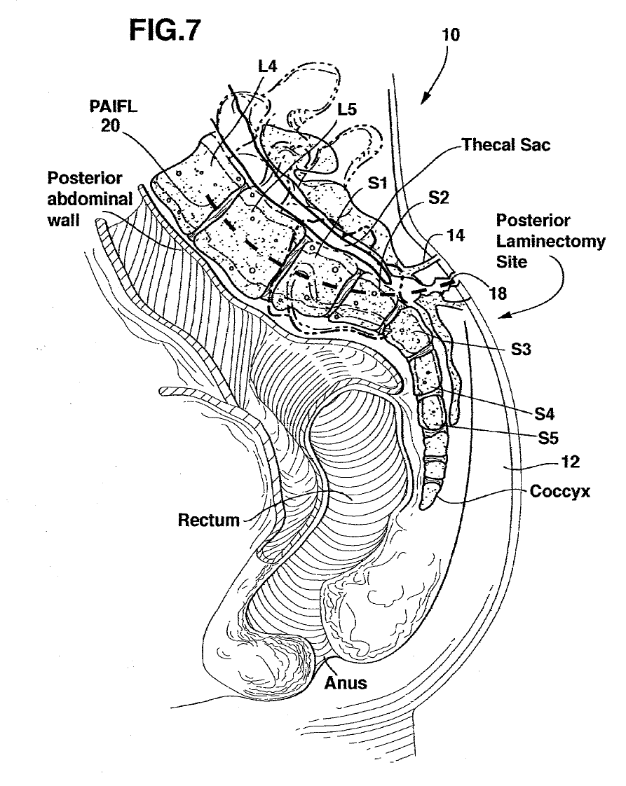

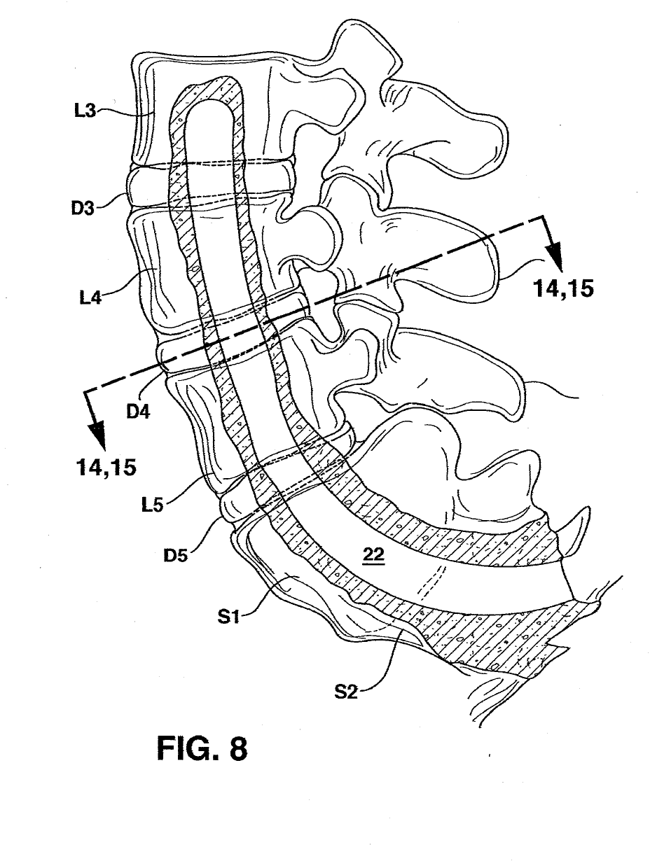

FIGS. 7 and 8 illustrate step S100 for forming the posterior percutaneous tract and the posterior TASIF axial bore 22 formed in step S200 and extending through sacral and lumbar vertebrae and intervening discs axially aligned with the visualized PAIFL of FIGS. 1 and 2 using a boring tool of the type described in more detail in the above-referenced '105 and '748 applications. The same steps can be employed to form a pilot hole of step S100 that can be enlarged in step S200. In this case, a small diameter bore forming tool (e.g. 3.0 mm diameter) is used to first bore a small diameter curved pilot hole following the imaginary, visualized PAIFL 20 through S1, L5 and L4 in step S100. Then, the boring tool is removed, and a guidewire having a threaded distal screw-in tip is advanced through the pilot hole and screwed into to the caudal end of the pilot hole and into cephalad portion of the L4 body. An over-the-wire bore enlarging tool having a flexible body capable of tracking the curved guidewire is fitted over the proximal end of the guidewire and manually or mechanically rotated and advanced along it in step S200. In this way, the small pilot hole diameter is enlarged to form the anterior TASIF axial bore 22 having a diameter e.g. a 10.0 mm diameter, and the enlarging tool is then removed.

It will be understood that the illustrated diameter of the posterior TASIF axial bore hole 22 relative to sizes of the vertebral bodies is merely exemplary, and that it is contemplated that the pilot hole and bore hole diameters can range from about 1-10 mm and 3-30 mm, respectively. Moreover, it will be understood that a plurality of such posterior TASIF axial bores 22₁ . . . 22ₙ can be formed in side by side or diverging relation generally aligned with the PAIFL.

In FIG. 7, the posterior surface of the sacrum is exposed in step S100 as described in the above-referenced '222 and '748 applications. The area of the patient's skin surrounding the incision site is surgically prepped, and the anus is excluded from the surgical field using adhesive drapes. The actual dermal entry site may be determined by the prone, preoperative CT scan or magnetic resonance imaging (MRI) study that maps the PAIFL. In step S100, an incision is made in the patient's skin over the posterior sacral surface of S2, and the subcutaneous tissue is separated to expose the posteriorly extending, bony ridge of the posterior sacral surface. A small laminectomy 14 is performed through the posterior ridge of the sacrum inferior. The thecal sac and nerve roots that are exposed by the laminectomy are gently retracted, and the terminal portion of the spinal canal is exposed.

An elongated drill shaft assembly (not shown) is axially aligned with the PAIFL. at the posterior target point so that the initial penetration of the sacrum is substantially at right angles to the exposed sacral surface. A drill guide for receiving the drill drive shaft assembly for drilling or boring a posterior TASIF axial bore 22 from S2 along the visualized PAIFL may optionally be attached to S2 and extended posteriorly through the exposed spinal canal and skin incision.

The progress of the drill bit is observed using conventional imaging equipment. As the elongated drill shaft assembly is extended anteriorly in the cephalad direction, a curvature is introduced in the cephalad segment of the posterior TASIF axial bore 22 as shown in FIG. 8. It is necessary to maintain the plane of curvature of the distal segment aligned to the curvature of the spine. In this way, the drill bit advances through the sacral vertebrae in the cephalad direction and toward the lumbar vertebral bodies while staying within the spongy, cancellous bone of each vertebral body. Theoretically, any number of vertebral bodies of the spine can be bored through in the cephalad axial direction. The cephalad end of the posterior TASIF axial bore 22 can terminate within a vertebral body or within a disc or disc space in either case providing an axial disc opening to an intervertebral disc for performing a discectomy and any ancillary procedures.

Anterior TASIF Axial Bore Formation:

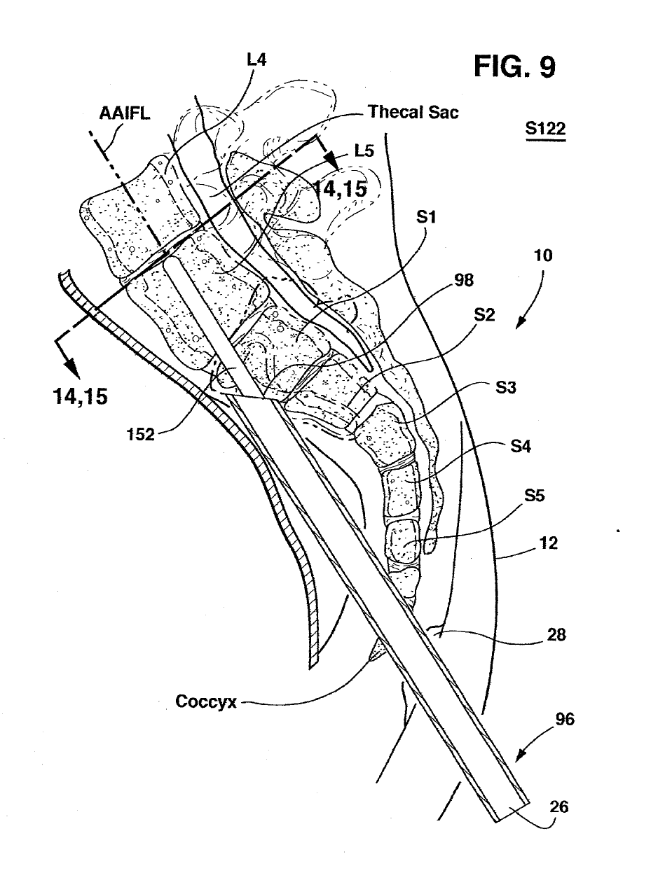

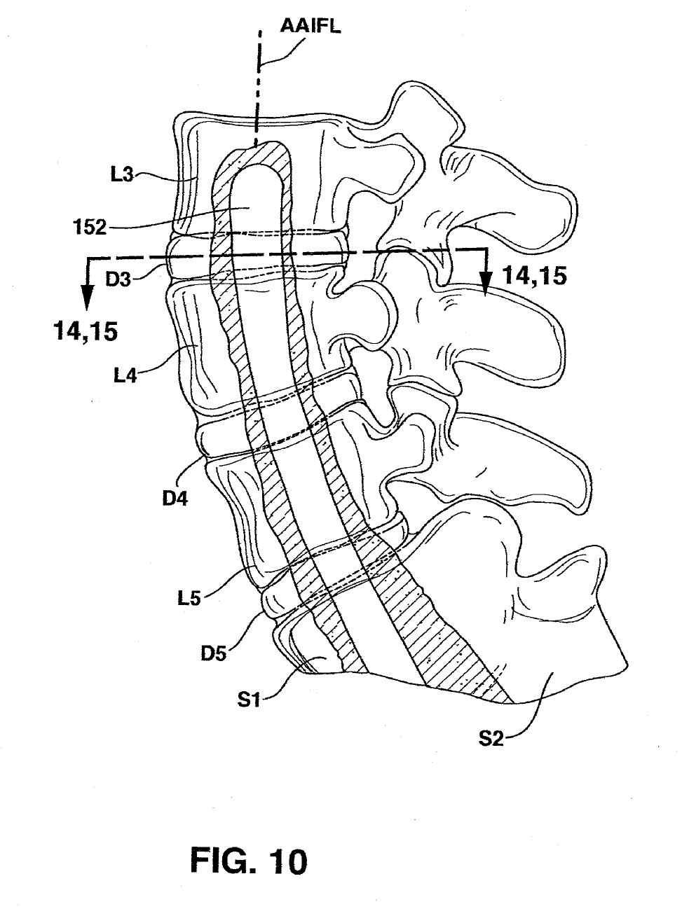

FIGS. 9 and 10 illustrate the anterior percutaneous tract formed in step S100 and the anterior TASIF axial bore 22 formed in step S200 and extending through sacral and lumbar vertebrae and intervening discs axially aligned with the visualized AAIFL of FIGS. 1 and 2 using a boring tool of the type described in more detail in the above-referenced '105 and '748 applications. The same steps can be employed to form a pilot hole of step S100 that can be enlarged in step S200 as described above. It will be understood that the illustrated diameter of the anterior TASIF axial bore hole 152 relative to sizes of the vertebral bodies is merely exemplary, and that it is contemplated that the pilot holes and bore hole diameters can range from about 1-10 mm and 3-30 mm, respectively. Moreover, it will be understood that a plurality of such anterior TASIF axial bores 152₁ . . . 152ₙ can be formed in side by side or diverging relation generally aligned with the AAIFL.

The anterior TASIF axial bore(s) can be relatively straight from the anterior target point into or through at least the caudal lumbar vertebrae and intervertebral discs. But, it may be desirable or necessary to form a curved anterior TASIF axial bore(s) particularly as the bore(s) is extended in the cephalad direction to maintain the plane of curvature of the cephalad segment of the TASIF axial bore(s) aligned to the curvature of the spine. In this way, the drill bit advances through the sacral vertebrae in the cephalad direction while staying within the spongy, cancellous bone of each vertebral body. Theoretically, any number of vertebral bodies of the spine can be bored through in the cephalad direction. The cephalad end of the posterior TASIF axial bore(s) 152 can terminate within a vertebral body or within a disc or disc space in either case providing an axial disc opening to an intervertebral disc for performing a discectomy and any ancillary procedures.

Diverging TASIF Axial Bore(s):

If a single anterior or posterior TASIF axial bore is to be made, it preferably is axially aligned with the respective visualized AAIFL or PAIFL as shown by TASIF axial bore 22 or 152 shown in FIG. 4. Plural anterior or posterior TASIF bores 22₁ . . . 22ₙ, or 152₁ . . . 152ₙ shown in FIG. 5 are in parallel or diverging alignment with the visualized AAIFL and PAIFL. Multiple anterior or posterior TASIF axial bores can be formed all commencing at an anterior or posterior target point of FIGS. 1-3 and extending in the cephalad direction with each TASIF axial bore diverging apart from the other and away from the visualized axial AAIFL and PAIFL. The diverging TASIF axial bores terminate at spaced apart locations in a cephalad vertebral body or in separate cephalad vertebral bodies or spinal discs if a discectomy of more than one spinal disc is to be performed.

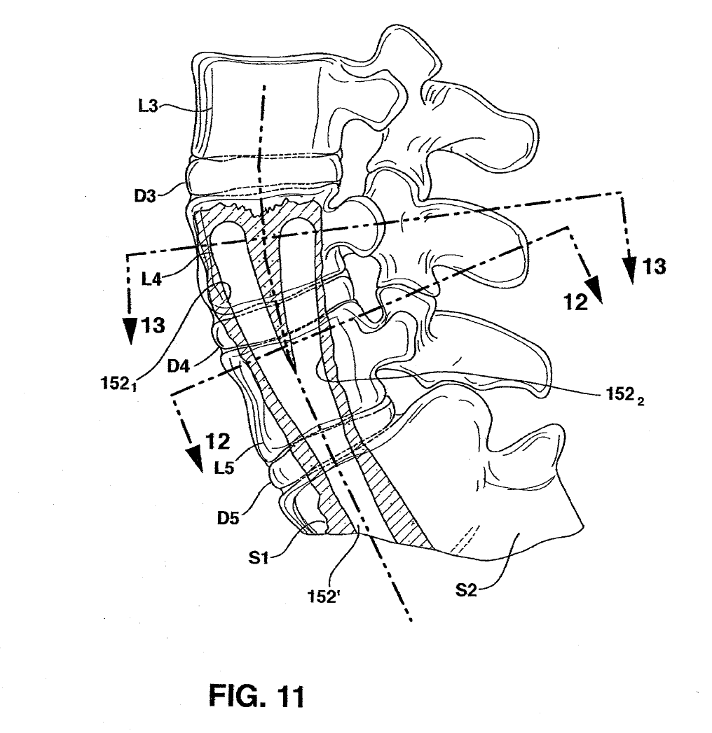

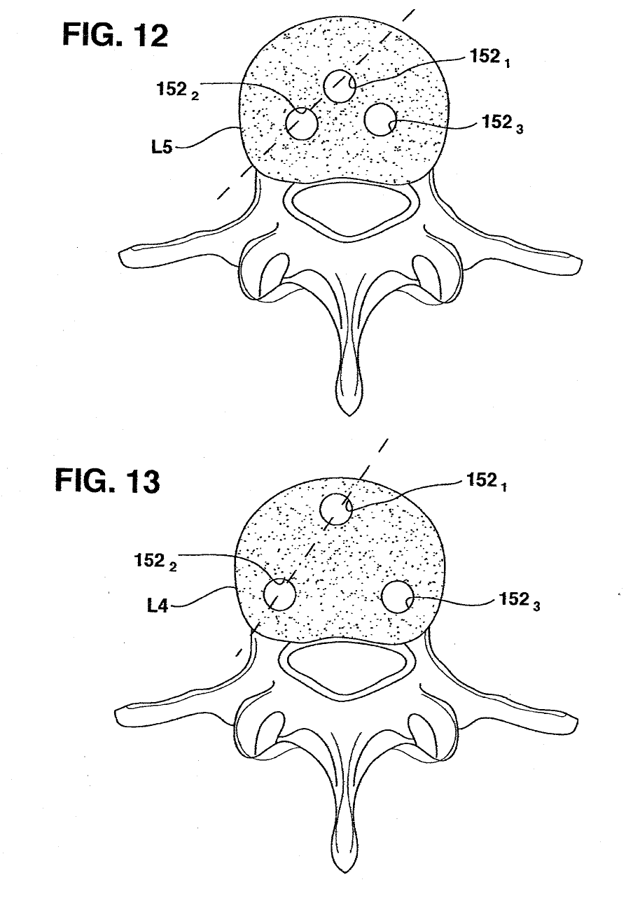

For example, FIGS. 11-13 depict a group of three anterior TASIF axial bores 152₁, 152₂, 152₃ that are bored from a common caudal entrance bore section 152′ starting at the anterior target point. The three anterior TASIF axial bores 152₁, 152₂, 152₃ extend in the cephalad direction generally following the curvature of the AAIFL but diverging outwardly to form a “tripod” of the diverging TASIF axial bores 152₁, 152₂, 152₃. The divergence from the common entry bore section can start in the sacral vertebra or in L5 or in L4 or in any other cephalad vertebral body that the bore extends into or through. The common caudal entrance bore section 152′ through S1, and traversing disc D5 and part of L4 can be larger in diameter than the diverging TASIF axial bores 152₁, 152₂, 152₃ to facilitate performing one or more discectomy as described further below. The diverging TASIF axial bores 152₁, 152₂, 152₃ can be extended further than shown in FIGS. 11-13. Diverging posterior TASIF axial bores can be formed in the same manner.

In accordance with the present invention, it may be preferable in certain cases to form only a single diverging TASIF axial bore, e.g., the TASIF axial bore 152₂ or 152₃, to locate an axial disc opening close to a herniated area of the spinal disc that intrudes upon a nerve root. Then, the discectomy instrument can be directed into the protruding disc annulus and/or nucleus to remove it and relieve the pressure applied to the nerve root.

Discectomy Instruments and Procedures:

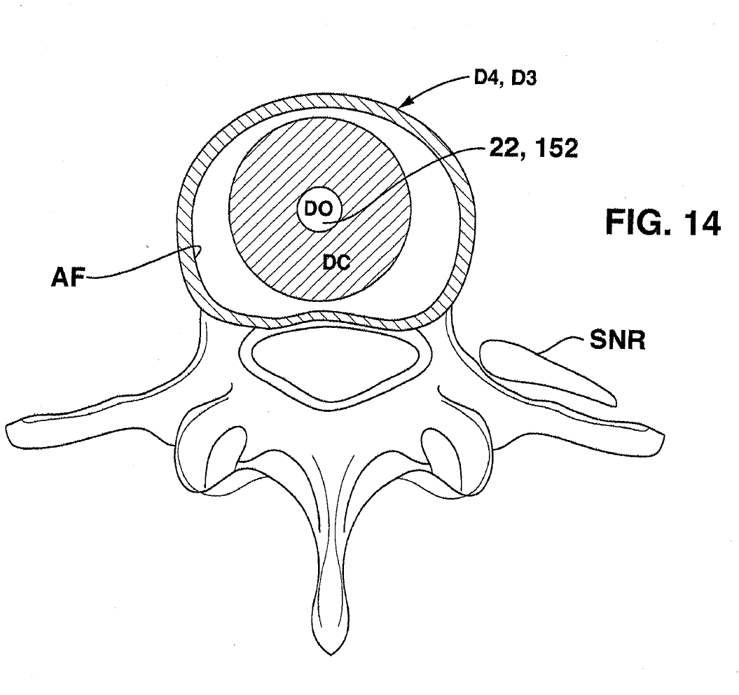

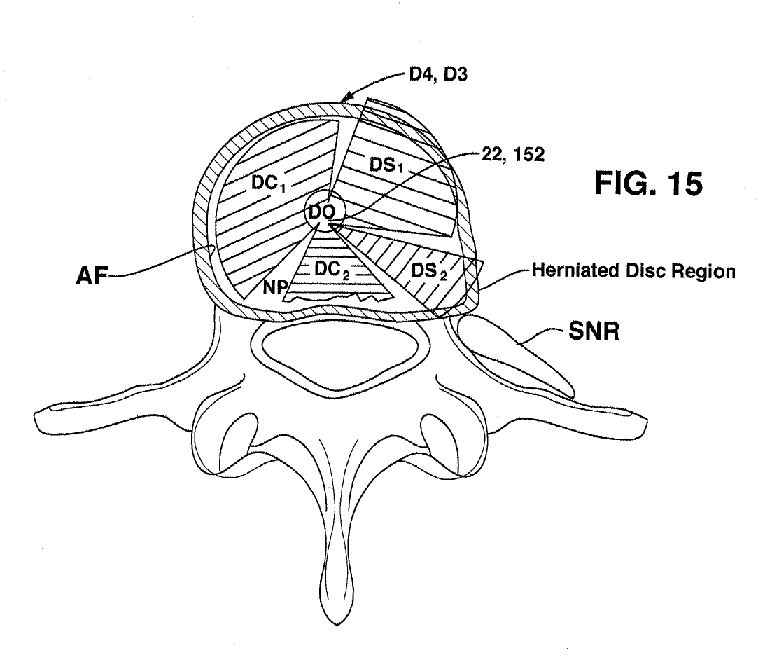

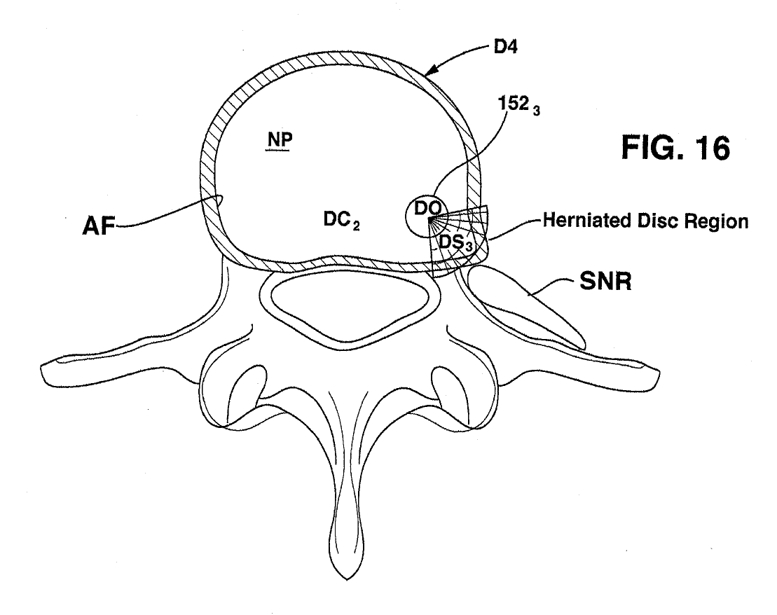

An above-described anterior or posterior TASIF axial bore 22, 152, et seq., is formed to extend into or through an intervertebral spinal disc where a discectomy is to be performed. FIGS. 14-16 illustrate a laterally sectioned spinal disc, e.g., D4 and D3, that can be accessed by a TASIF axial bore 22, 152 to make an axial disc opening DO into or through the spinal disc. Then, a discectomy instrument or instruments of the present invention can be introduced into the nucleus NP and used to excise or desiccate all or selected portions of the nucleus NP and the annulus AF to form a disc cavity within the annulus AF or a disc space encompassing part or all of the annulus AF. The disc cavity DC within the annulus AF can be generally circular as shown in FIG. 14 or can be an arcuate disc cavity DC₁, or DC₂, as shown in FIG. 15 or DC₃ as shown in FIG. 16. Alternatively, the annulus AF can be excised to form a disc space DS₁ or DS₂ as shown in FIG. 15. It will be understood from the following description of the discectomy instrument that much of the annulus AF can be removed to form an irregular shaped disc space and that a wide variety of disc cavity shapes can be formed.

A portion of the spinal disc D4 is shown to be herniated in a herniated disc region and bulging toward one of the spinal nerve roots SNR in FIGS. 15 and 16. In FIG. 15, the arc or pie shaped disc space DS₂ is formed extending from the generally axially aligned axial bore 22, 152. In FIG. 16, the axial bore 22, 152 is formed to diverge generally toward the herniated disc region as axial bore 1523 diverges in FIGS. 11-13. The pie-shaped disc space DS₃ can then be formed in that region. In both cases, it may or may not be necessary to compromise the annulus AF if it is not yet rent, and it may or may not be necessary to remove more of the annulus AF and/or nucleus NP.

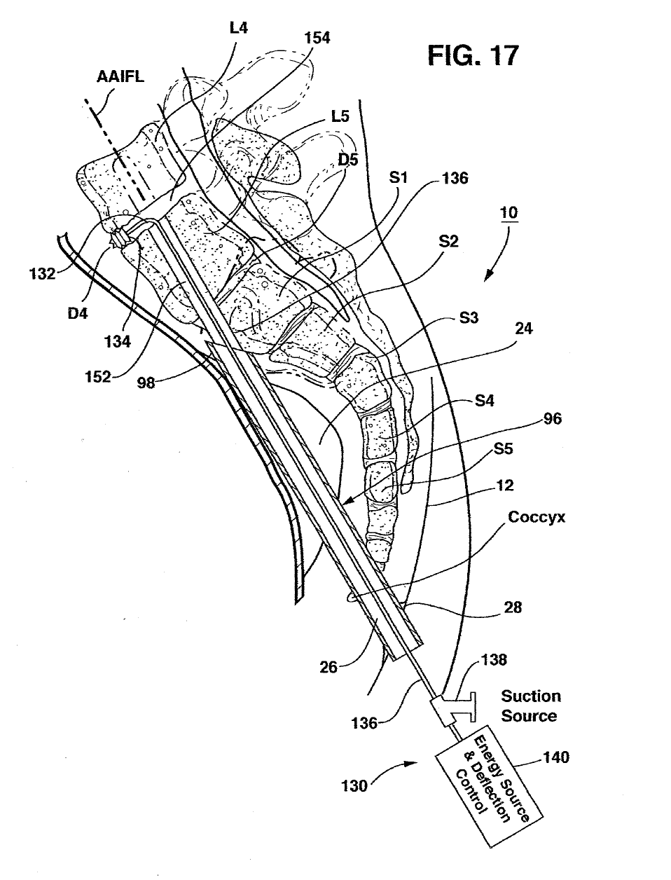

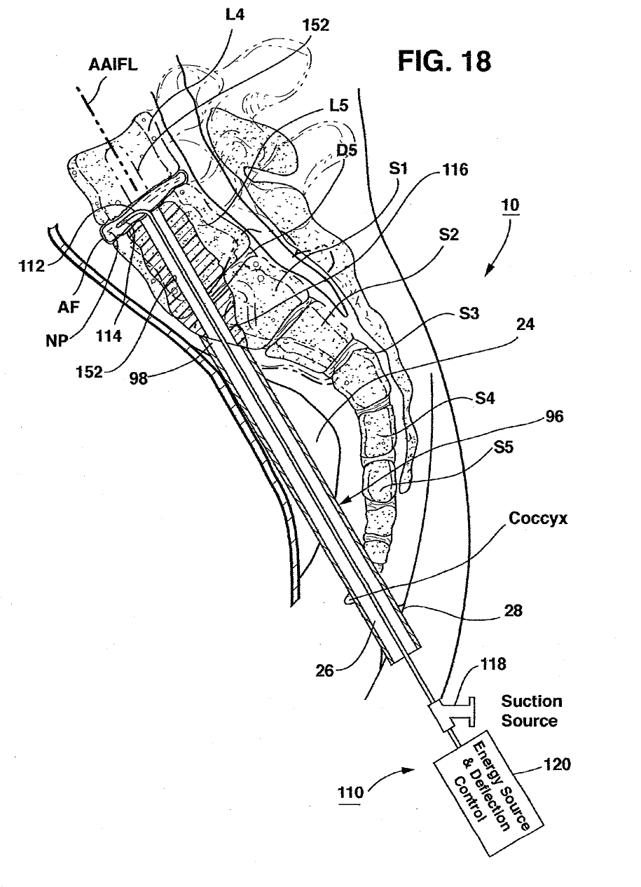

For convenience of illustration, the discectomy procedures illustrated in FIGS. 17 and 18 are described as follows as being performed through an anterior percutaneous tract formed using an anterior tract sheath 96 and TASIF axial bore 152 providing an axial disc opening in at least a caudal endplate of a spinal disc. But, it will be understood that these illustrated discectomy procedures and discectomy instruments may be performed through and used in a posterior percutaneous tract and TASIF axial bore 22 of any of the above-described types.

In anterior discectomy procedures, the anterior TASIF axial bore 152 is formed, as described above, through the use of anterior tract sheath 96 that inserted earlier through the presacral space 24 from a skin incision 28 to the anterior target point of the anterior surface of sacral vertebra S1 that defines the percutaneous tract 26. The shaped end 98 of the anterior tract sheath 96 is aligned with the anterior surface of the sacral vertebra S1 during step S1100. The shaped end 98 may be formed with attachment teeth or threads to fix it to the sacral bone. It will be understood that the discectomy instruments of the present invention may be performed through the lumen of such a tract sheath 96 or simply through a defined anterior tract 26 extending through the pre-sacral space 24 to axially access the vertebrae.

As described above, the complete discectomy procedures conducted in the past have been done through lateral exposure of the disc that presents a number of problems that are eliminated by the present invention.

First Discectomy Instrument Type:

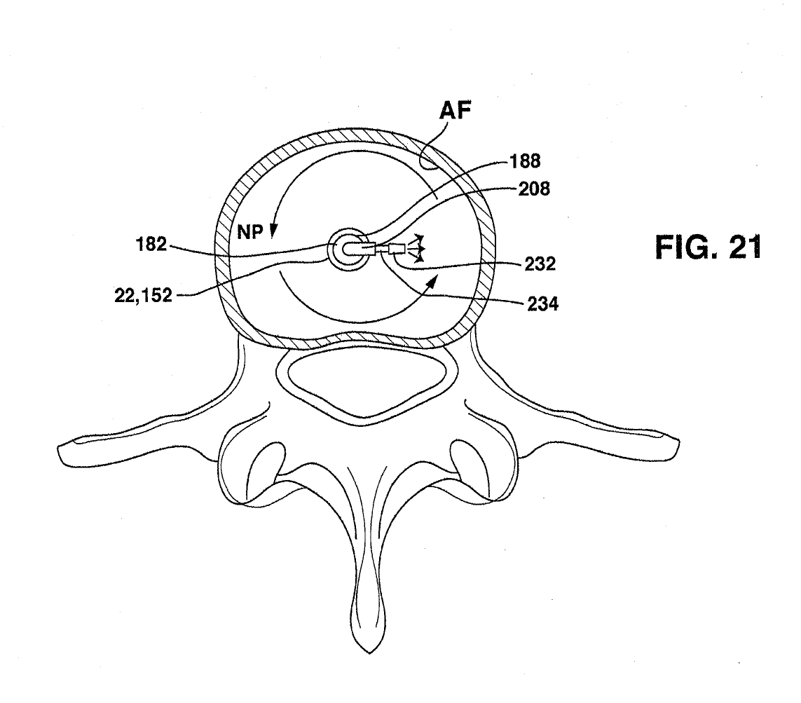

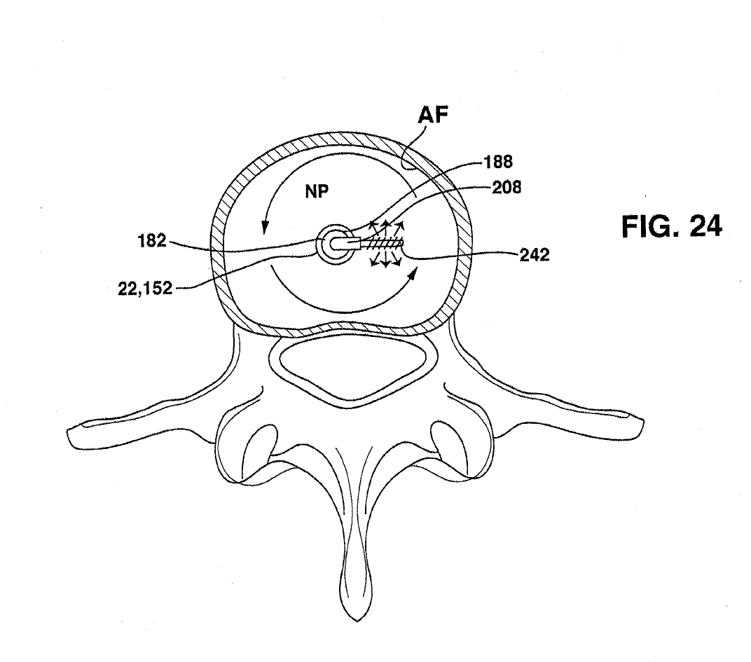

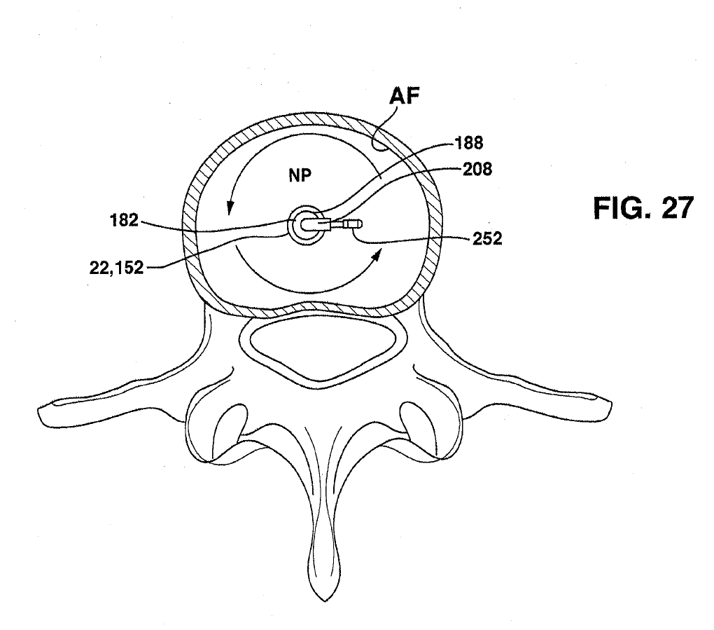

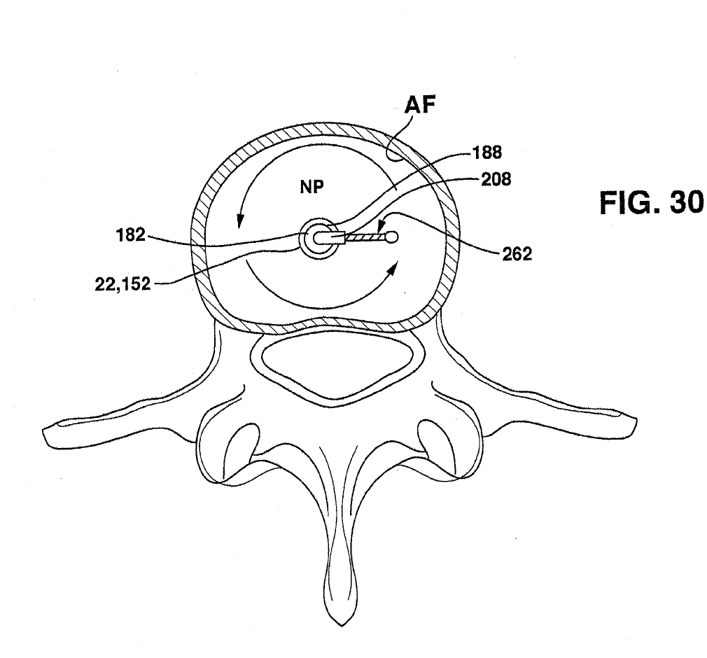

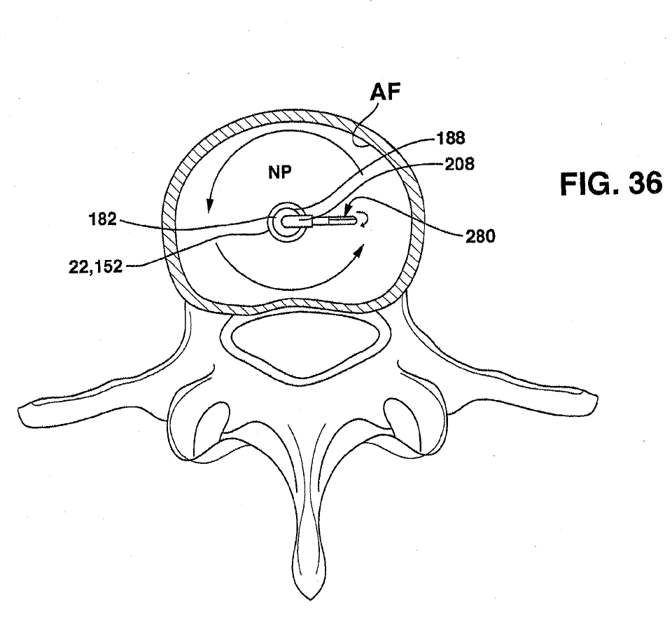

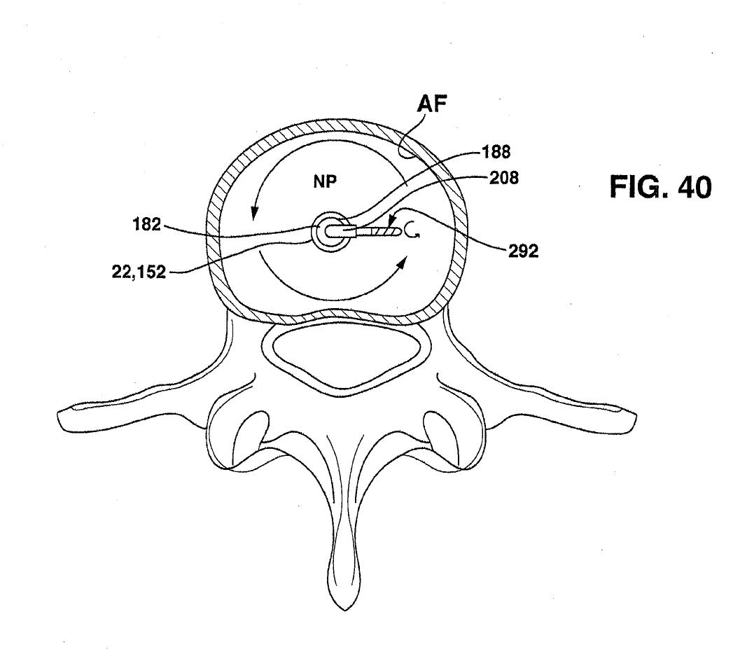

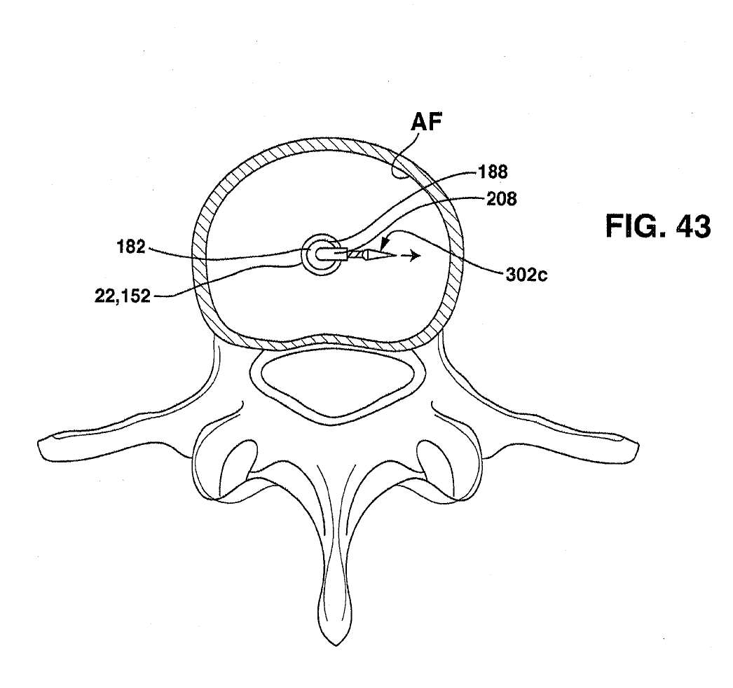

A first preferred type of discectomy instrument comprises a cutting head that is mounted to the distal end of a flexible discectomy instrument shaft that can traverse the short radius bend at the axial disc opening laterally into the annulus as shown in FIGS. 17-46. The cutting head is axially aligned with the discectomy instrument shaft when unrestrained and when advanced through the TASIF axial bore 22, 152 or a discectomy sheath lumen. But, a bend can be formed in the discectomy instrument shaft proximal to the cutting head to allow the deflection of the cutting head laterally and radially away from the axial disc opening. Then, as the cutting head is extended further laterally toward the annulus, the discectomy instrument shaft bends as it passes through the axial disc opening.

FIGS. 17 and 18 illustrate a first variation of the first type of discectomy instrument wherein the deflection of the distal cutting head is effected using an externally manipulatable and internally disposed mechanism for selectively forming a bend in the discectomy instrument shaft proximal to the cutting head to pass the cutting head into the disc nucleus from the axial bore.

FIG. 17 illustrates, in a partial cross-section side view, one manner of performing a discectomy of a spinal disc, e.g., D4, effected through a TASIF axial bore 152 to enable fusion of the vertebral body endplates of lumbar vertebrae L4 and L5 directly together or to provide a disc space for receipt of a pre-formed artificial disc implant that mimics the function of a patent spinal disc. The illustrated complete discectomy procedure involves more or less complete excision of the intervertebral disc D4 including the nucleus, and at least a portion of the annulus and can include excision of the cartilaginous endplates adhered to the opposed cortical bone endplates of the cephalad and caudal vertebral bodies, and, optionally, the vertebral periosteum, cortical endplate bone and cancellous bone to a desired depth and shape. Distraction is applied to the lumbar vertebrae L4 and L5 by suitably supporting the patient's body to maintain the disc space for fusion and/or implantation of an artificial disc implant. The excised materials are withdrawn from the disc space and the fusion materials or artificial disc implant is introduced to the disc space through the TASIF axial bore 152.

The TASIF axial bore 152 either terminates within the spinal disc to be removed or extends into a vertebral body cephalad to that spinal disc in the event that an axial spinal implant or bone growth material is to be inserted into the TASIF axial bore bridging the excised disc space. FIG. 17 depicts the TASIF axial bore 152 in solid lines terminating in the disc D4 and in broken lines extending into vertebral body L4.

Then, a discectomy instrument 130 is inserted through the axially aligned anterior tract 26 defined by the lumen of the anterior tract sheath 96. The discectomy instrument 130 is of a first type wherein the cutting head is remotely manipulatable to be advanced through the TASIF axial bore 152 and to then be deflected laterally into the nucleus of the spinal disc. The discectomy instrument 130 is formed like a flexible atherectomy catheter for fragmenting and removing obstructions in blood vessels using a cutting head 134 to fragment the disc material and to scrape away cortical and cancellous bone and aspiration with saline flushing to remove the fragments from the disc space. The cutting head 134 is mounted into a deflectable or steerable distal end section 132 of discectomy instrument shaft 136 extending through the TASIF axial bore 152 and anterior tract 26 from an externally disposed energy source and deflection control 140. The distal end section may be angularly deflected using a deflection mechanism, e.g., a pull wire within a pull wire lumen of the elongated, flexible, discectomy instrument shaft 136 and a proximal pull wire control of the proximal guiding and cutting mechanism 140 coupled thereto. The cutting head 134 may be pulled back and forth laterally and/or swept in a 360° arc about the axial bore 152 to traverse and excise selected symptomatic portions of or the entire spinal disc D4 and to cut away layers of bone from the endplates of vertebral bodies L4 and L5 by manipulation of the proximal end portion of the discectomy instrument shaft 136 extending from the skin incision 28. The discectomy cutting head 134 and tool shaft 136 are shown schematically and not necessarily to scale to one another or to the TASIF axial bore 152.

The cutting head 134 in this example is a mechanical screw thread that can be selectively covered in whole or part by a sheath (not shown) for exposing the end or a lateral portion of the cutting screw thread in the manner of the augur disclosed in the above-referenced '884 patent for example. The cutting head 134 is attached to a drive shaft extending through a drive shaft lumen of the tool shaft 136 to a drive motor for rotating the drive shaft and cutting head 134 and a deflection control for operating the pull wire or the like for deflecting the distal section 132, both included in energy source and deflection control 140. Preferably an aspiration lumen is included within the discectomy instrument body 136 with a distal opening adjacent to the cutting head 134 and terminating proximally at a side suction port 138 adapted to be coupled to a source of suction to aspirate the fragments of the disc from disc space 154. A saline flush lumen and supply can also be incorporated within the discectomy instrument body to flush blood and excised fragments for aspiration.

The operation and movement of the cutting head about the spinal disc D4 is preferably observed employing MRI, fluoroscopy or other radiographic visualization techniques. An endoscopic visualization or the disc space 154 could also be employed using a separate or incorporated deflectable tip endoscope for illumination and observation of the site.

The resulting disc space 154 can either be substantially disc-shaped with more or less planar opposed sides having a height in the range of about 8 mm to about 14 mm, a lateral width of from about 26 mm to about 32 mm, and an anterior-posterior width of from about 22 mm to about 30 mm. However, the disc space 154 can be selectively enlarged into a convex disc shape extending caudally into the cancellous bone of caudal vertebral body L5 and/or in the cephalad direction into the cancellous bone of cephalad vertebral body L4. This disc space shaping forms a pocket that helps to confine a spinal disc implant inserted into the prepared disc space 154 or bone growth materials dispensed into the prepared disc space 154.

A fusion of vertebral bodies D4 and D5 or the implantation of an artificial spinal disc into the disc space 154 through the anterior TASIF axial bore 152 and percutaneous tract 26 may be undertaken after the disc space is cleared of debris.

Moreover, the TASIF axial bore can be filled with an axial spinal implant that provides internal stabilization, alignment, and reinforcement of the spinal motion segment. This therapeutic procedure of the present invention can be advantageously conducted without any injury to any ligaments, muscles and facet joints of the spinal motion segment.

In many instances, it is preferable to perform a partial discectomy or disc decompression where the annulus is left intact and relief from pain caused by a rupture or swelling against the spinal cord or nerves is sought. As described above, the partial discectomy procedures conducted in the past have been done through lateral exposure of the disc and perforation of the annulus that presents a number of problems that are eliminated by the present invention. In accordance with the present invention the performance of a partial discectomy does not involve compromising or breaching the annulus and only the nucleus or a portion of the nucleus is removed to form a disc cavity

In this aspect of the present invention, the anterior or posterior TASIF axial bore 152 or 22 is formed in the manner described above and terminates at an axial disc opening to the nucleus of the disc to be treated or optionally extends into the cephalad vertebral body to facilitate fusion of the vertebral bodies following a discectomy. Discectomy instruments can be introduced through the TASIF axial bore and percutaneous tract into the nucleus to fragment or desiccate all or part of the nucleus, including any projecting into the annulus or from a fissure in the annulus, and create a void or disc cavity within the annulus and the cartilaginous endplates. Any fissures or other damage or weakening of the annulus can be treated from within the created void in a manner described in the above-referenced '149 patent, for example. In a simple decompression, the entry into the nucleus and the TASIF axial bore can then be closed with a simple elongated axial spinal implant or a shorter plug formed of a bone growth material or another bio-compatible material or bone cement. Alternatively, a disc augmentation can be performed before closure as described further below.

FIG. 18 illustrates, in a partial cross-section side view, one manner of performing a partial discectomy of a spinal disc to remove at least a portion of the nucleus NP effected through a TASIF axial bore 152 while leaving the annulus AF intact as shown in FIG. 14, for example. A discectomy instrument 110 is inserted through the axially aligned anterior tract 26 defined by the lumen of the anterior tract sheath 96 and the TASIF axial bore 152. The discectomy instrument 110 is of a first type wherein the cutting head is remotely manipulatable to be advanced through the TASIF axial bore 152 and to then be deflected laterally into the nucleus of the spinal disc. The discectomy instrument 110 is formed like a flexible atherectomy catheter for fragmenting and removing obstructions in blood vessels using a cutting head 114 to fragment or desiccate the disc material and aspiration with saline flushing to remove the fragments or by-products from the void or cavity created in the annulus. The cutting head 114 is mounted into a deflectable or steerable distal end section 112 of discectomy instrument shaft 116 extending through the TASIF axial bore 152 and anterior tract 26 from an externally disposed energy source and deflection control 140.

For example, the cutting head 114 can comprise a cutting wire that is projected from a side opening in the distal end section 112 as a loop that is rotated to slice sections of the nucleus into fragments that are aspirated through a lumen of the discectomy instrument shaft 116. The distal end section 112 may be angularly deflected using a deflection mechanism, e.g., a pull wire within a pull wire lumen of the elongated, flexible, discectomy instrument shaft 116 and a proximal pull wire control of the proximal guiding and cutting mechanism 120 coupled thereto. The cutting head 114 may be pulled back and forth laterally and/or swept in a 360° arc about the axial bore 152 to traverse and excise selected symptomatic portions of the spinal disc D4 including the internally disposed nucleus NP and to cut away an portion of it that is extruded through a fissure in the annulus AF by manipulation of the proximal end portion of the discectomy instrument shaft 116 extending from the skin incision 28. The discectomy cutting head 114 and tool shaft 116 are shown schematically and not necessarily to scale to one another or to the TASIF axial bore 152.

The retractable/expandable cutting wire of exemplary cutting head 114 can be extended out of and retracted back into the cutting head 114. The distal section 112 is attached to a drive shaft extending through a drive shaft lumen of the tool shaft 116 to a drive motor included in energy source and deflection control 120 for rotating the drive shaft and cutting head 114. A deflection control for operating the pull wire or the like for deflecting the distal section 112 is also included in energy source and deflection control 120. Preferably an aspiration lumen is included within the discectomy instrument body 116 with a distal opening adjacent to the cutting head 114 and terminating proximally at a side suction port 118 adapted to be coupled to a source of suction to aspirate the fragments of the nucleus from the cavity formed inside the annulus of spinal disc D4. A saline flush lumen and supply can also be incorporated within to flush excised fragments for aspiration.

The operation and movement of the cutting head 114 about the interior of spinal disc D4 is preferably observed employing MRI, fluoroscopy or other radiographic visualization techniques. An endoscopic visualization or discoscopy of the cavity formed within the annulus AF could also be employed using a separate or incorporated deflectable tip endoscope for illumination and observation of the site. Weakened or damaged sections or fissures in the annulus AF can be visually detected in this way.

In addition, further instruments and materials can be introduced into the cleared space to maintain distraction spacing of the vertebral bodies D4 and D5 and to make repairs to weakened or damaged sections or fissures of annulus AF. Such repairs can be made by heat treatment or by the application of a biocompatible patching material, such as a fibrin glue, against the interior surface of the annulus AF by inflation of a balloon within the cavity as described in the above-referenced '149 patent.

The partial discectomy can be advantageously conducted without any injury to any ligaments, muscles and facet joints of the spinal motion segment and avoids compromising the annulus. The disc cavity can be circular as shown in FIG. 14 or comprise one or more arc or pie-shaped segments as shown in FIG. 15.

The discectomy instruments 110 and 130 and the remaining described discectomy instruments of the present invention could be inserted through the TASIF axial bore 22, 152, but it is believed desirable to provide a straight or curved discectomy sheath 180 that can be fitted therein to extend from outside the patient to the axial disc opening. The discectomy sheath 180 illustrated in the remaining figures provides a smooth interior surface of the sheath lumen 182 that enables advancement of the discectomy instrument and cutting head or a deflection catheter 200 therethrough. The discectomy sheath 180 and the discectomy sheath lumen 182 extend from discectomy sheath proximal and distal ends 186 and 188. The discectomy sheath 180 is also provided with a side port 184 distal to a proximal end fluid tight seal (not shown) penetrable by the discectomy instrument shaft or a deflection catheter. The side port 184 can be selectively coupled to a fluid source for delivering irrigation fluid through the sheath lumen 182 into the spinal disc or to an aspiration pump for aspirating fluid through the sheath lumen 182 to alternately irrigate an aspirate the operative space. Or, the irrigation fluid can be delivered through a separate irrigation fluid delivery lumen of the sheath 182 or the discectomy instrument shaft or the deflection catheter lumen.

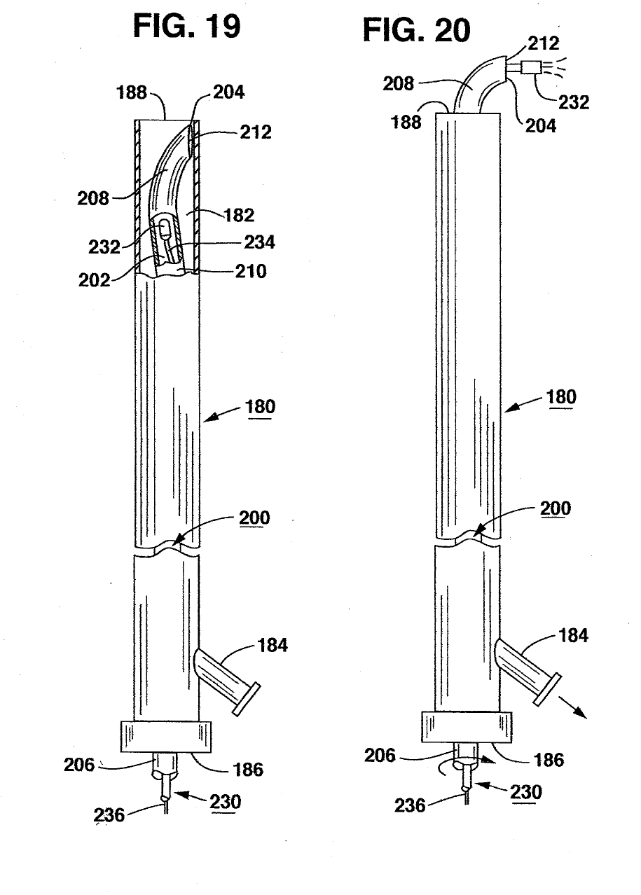

In a second variation of this first type of discectomy instrument illustrated in the embodiments of FIGS. 19-46, the deflection of the cutting head with respect to the discectomy instrument shaft is enabled by a deflection catheter 200 having a deflection catheter lumen 202 extending between a deflection catheter proximal end 204 and a deflection catheter distal end 206. A distal portion 208 of the deflection catheter 200 is angled with respect to a proximal portion 210 of the deflection catheter 200 to orient the deflection catheter lumen distal end opening 212 at about 90° with respect to the deflection catheter lumen 202 in the proximal portion 210.

It will be understood that the particular discectomy instruments depicted in FIGS. 19-43 exemplify energy emitting or mechanical cutting heads that could be substituted for the mechanical cutting heads of discectomy instruments 110 and 130 described above so that these cutting heads could be deflected by the pull wire deflection mechanism rather than the deflection catheter 200. For convenience, however, these discectomy instruments will be described in the context of use of the deflection catheter 200 as illustrated by these figures.

In use, in each of the following embodiments of FIGS. 19-46, the deflection catheter 200 is extended either through the discectomy sheath lumen 182 within the TASIF axial bore or directly through the TASIF axial bore and into the axial disc opening to locate the distal portion 208 within the nucleus with the lumen distal end opening 212 facing toward the annulus. In each case, the discectomy instrument is already disposed within the deflection catheter lumen 202 during its advancement through the discectomy sheath lumen 182. The distal cutting head is disposed either in the proximal, straight portion or fitted within the lumen 202 within angled distal portion 208 of the deflection catheter 200. Then, the discectomy instrument shaft proximal end is advanced into the deflection catheter lumen 202 to advance the cutting head through the angled distal portion 208 and out of the lumen distal end opening 212 laterally and radially into the nucleus and toward or through the annulus. The discectomy instrument and deflection catheter 200 are then rotated either in an arc or through 360°, in some cases by hand and in other cases by a drive motor at a particular rpm. In certain cases, the cutting head is itself rotated by a drive motor.

Each embodiment of FIGS. 19-46 is depicted in a partially cut away side view illustrating the discectomy instrument and deflection catheter 200 within the discectomy sheath 180, a side view of the discectomy instrument cutting head and the angled distal portion extending from the sheath distal end 188, and a top view of the discectomy instrument cutting head and the angled distal portion extending from the sheath distal end 188 laterally and radially into the nucleus NP of a spinal disc. It will be understood that in each case, the cutting heads can be extended laterally and radially into or through the annulus AF.

FIGS. 19-21, 22-24 and 25-27 illustrate embodiments of energy emitting cutting heads fixed to the discectomy instrument shaft distal end including optical laser light emitters, resistance heating elements or electrocautery elements or the like that are energized via an external energy source coupled to proximal ends of conductors extending through the shaft. The discectomy instrument shaft and deflection catheter are rotated to sweep the energy emitter through the nucleus in selected arcs or in full rotation to desiccate the nucleus as the shaft is advanced from the shaft proximal end to advance the energy emitter laterally toward or through the annulus as illustrated in FIGS. 14-16, for example. The physician can monitor the extent of desiccation that is achieved by the emitted energy using a separate discoscopy instrument inserted through the axial bore or discectomy sheath lumen or such a the discoscopy instrument can be incorporated into the discectomy instrument shaft.

In FIGS. 19-21, the energy emitter 232 of discectomy instrument 230 emits laser light energy and is attached at the distal end of a discectomy instrument shaft 234 that extends through the deflection catheter lumen 202 to a discectomy instrument proximal end (not shown) that is located outside of the patient's body. Visible or near infra-red laser light is conducted from a conventional external laser source (not shown) through an optical fiber 236 encased within the discectomy instrument shaft 234. The laser energy source is switched on and off by the physician to selectively emit energy into the NP or AF that desiccates the tissue it strikes.

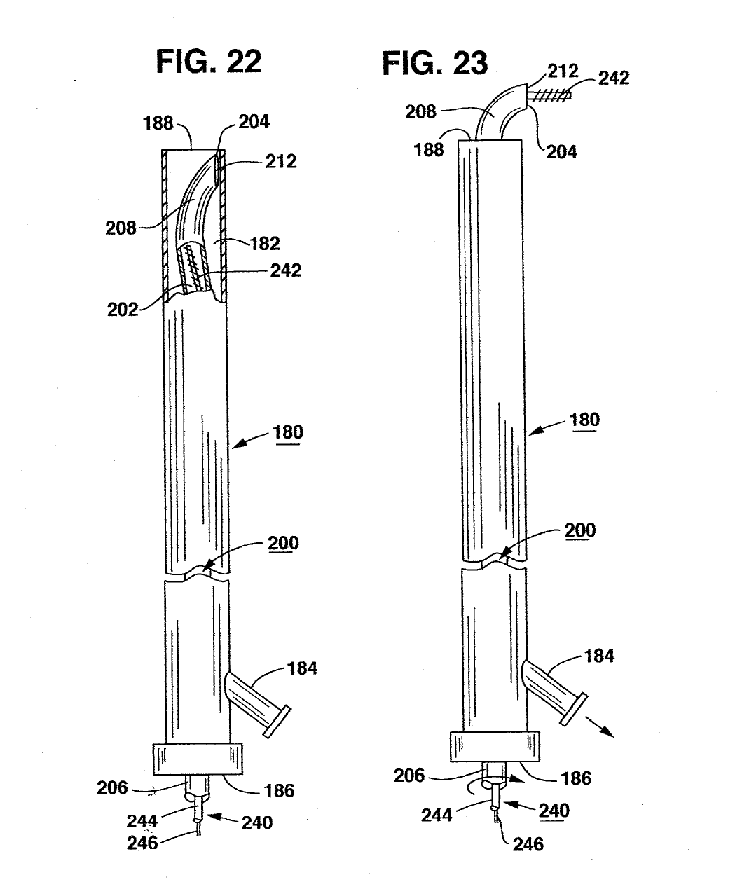

In FIGS. 22-24, the energy emitter 242 of discectomy instrument 240 emits thermal or heat energy and is attached at the distal end of a discectomy instrument shaft 244 that extends through the deflection catheter lumen 202 to a discectomy instrument proximal end (not shown) that is located outside of the patient's body. Electrical conductors 246 from the heat energy emitter 242 extend through the shaft 244 and are connected via a switch to a conventional external energizing source (not shown) that is opened and closed by the physician to selectively heat the energy emitter, whereby the heat is conducted into the adjacent NP or AP and desiccates the tissue. The thermal energy emitter 242 preferably comprises a resistance heating coil wound over a tubular insulating sheath. One of the electrical conductors 246 extends through the insulating sheath and is connected to the distal tip end of the resistance heating coil, whereas the other of the two conductors 246 is connected to the proximal end of the resistance heating coil.

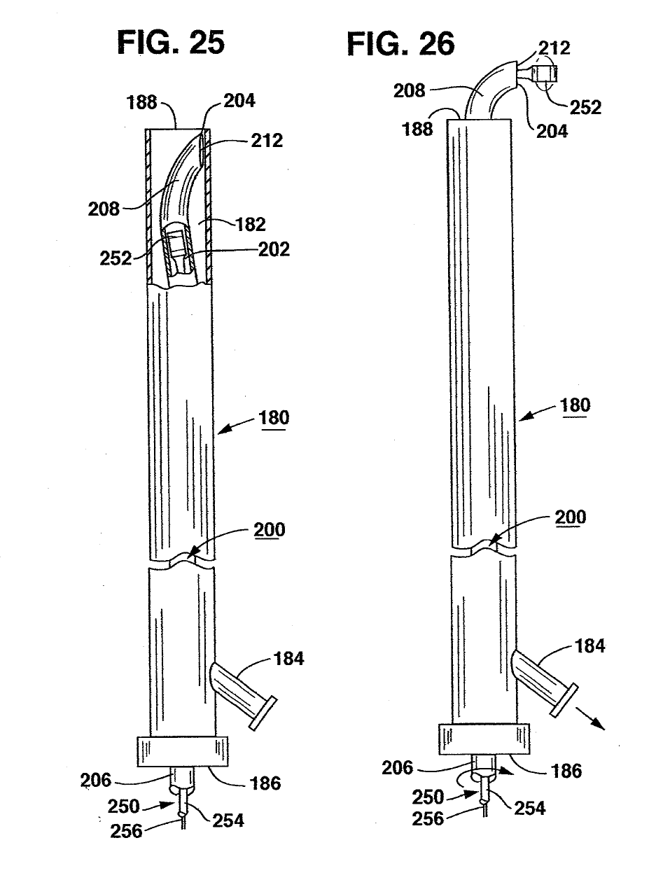

In FIGS. 25-27, the energy emitter 252 of discectomy instrument 250 emits electrocautery energy and is attached at the distal end of a discectomy instrument shaft 254 that extends through the deflection catheter lumen 202 to a discectomy instrument proximal end (not shown) that is located outside of the patient's body. Electrical conductors 256 from a pair of spaced apart ring electrodes forming the electrocautery energy emitter 252 extend through the shaft 254 and are connected via a switch to a conventional external energizing source (not shown) that is opened and closed by the physician to selectively emit electrocautery energy into the NP or AF. While bipolar electrocautery ring-shaped electrodes are shown, it will be understood that the electrodes may take any convenient shape. It will also be understood that the electrocautery energy emitter 252 can comprise a single unipolar electrode that is employed with a remote electrode in contact with the patient's body to apply electrocautery energy to the NP or AF in a unipolar mode.

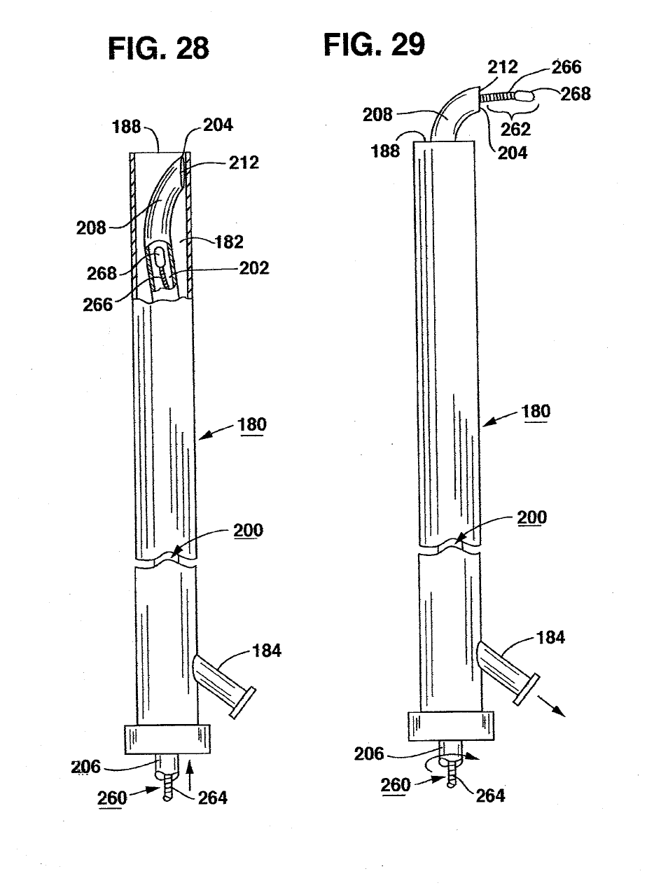

FIGS. 28-46 illustrate embodiments of mechanical cutting heads fixed to the discectomy instrument shaft distal end that cut or macerate or otherwise fragment the NP or AF tissues they are applied against.

Mechanical cutting tools usable in this variation of the first type of discectomy instrument include one or more flexible cutting wire having a fixed end fixed to a discectomy instrument shaft and a free end optionally having a weight at the free end. In FIGS. 28-30, a discectomy instrument 260 is depicted that has a mechanical cutting head 262 comprising a distal portion 266 of a flexible coiled or braided wire discectomy instrument shaft 264 and a weight 268. The angled distal portion 208 of the deflection catheter 200 is advanced through the disc opening, and the cutting head 262 is extended laterally as the assembly of the discectomy instrument 260 and the deflection catheter 200 are rotated at their proximal end by drive motor. The centrifugal force causes the cutting head 262 to cut through the NP to form a circular disc cavity as shown in FIG. 14. A portion of the AF can also be excised in this manner if the axial bore diverges toward it to form a DO closer to the AF section of interest than to other areas of the spinal disc.

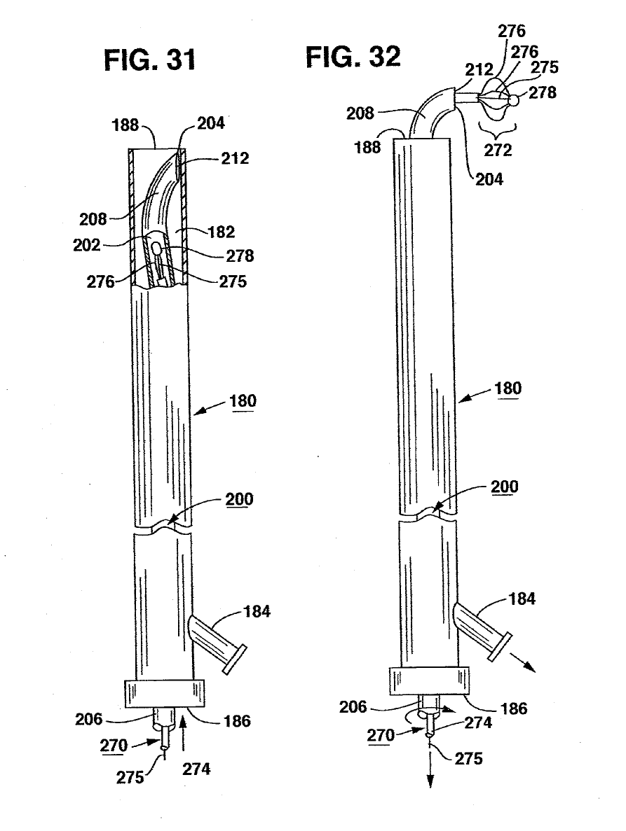

Further types of mechanical cutting tools shown in FIGS. 31-40 can also be employed in both variations of the first type of discectomy instrument, wherein the cutting head is movable with respect to the discectomy instrument shaft to apply cutting force against the AF or NP. The discectomy instrument comprises a discectomy instrument shaft lumen extending between discectomy instrument shaft proximal and distal ends of the discectomy instrument shaft, a rotatable or outwardly extendable cutting element, and a mechanism extending through the shaft lumen to the cutting element for rotating it or for deploying it.

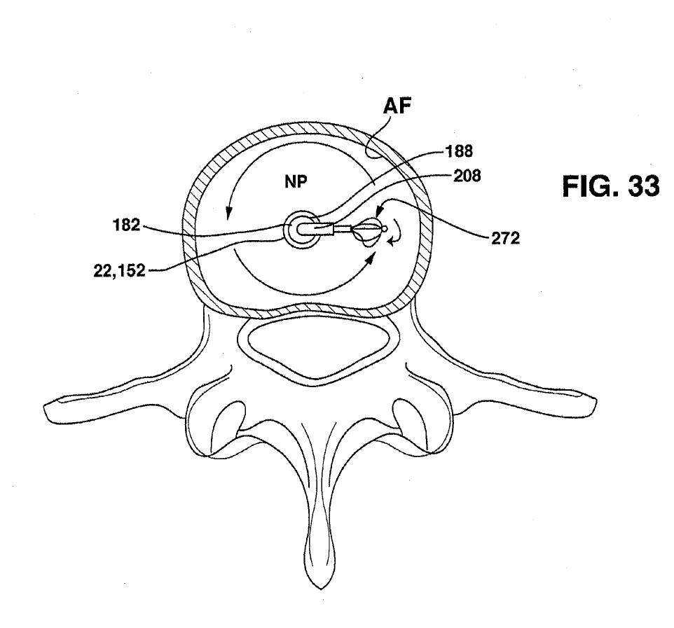

In one variation depicted in FIGS. 31-33, the discectomy instrument 270 comprises a discectomy instrument shaft 274 supporting a distally extending cutting head 272 formed of a plurality of cutting wires 276 extending from the shaft distal end to a common connection at a cutting head distal end 278. The discectomy instrument shaft 274 is formed with a shaft lumen extending between the discectomy instrument shaft proximal and distal ends. A retraction or pull wire 275 within the discectomy instrument shaft lumen is attached at its distal end to the cutting head distal end 278. The cutting wires 276 are formed of spring wire that is normally straight when the pull wire 275 is slack as shown in FIG. 31 but that can be bowed outward as the cutting head distal end 278 is retracted proximally when the pull wire 275 is retracted as shown in FIGS. 32 and 33. The degree of outward bowing can be controlled from the proximal end of the discectomy instrument shaft 274, and the pull wire 275 can then be locked with the discectomy instrument shaft 274. Then, the assembly of the discectomy instrument 270 and the deflection catheter 200 are rotated at their proximal end by drive motor. The centrifugal force causes the bowed out cutting wires 276 to cut through the NP to form a circular disc cavity as shown in FIG. 14. A portion of the AF can also be excised in this manner if the axial bore diverges toward it to form a DO closer to the AF section of interest than to other areas of the spinal disc.

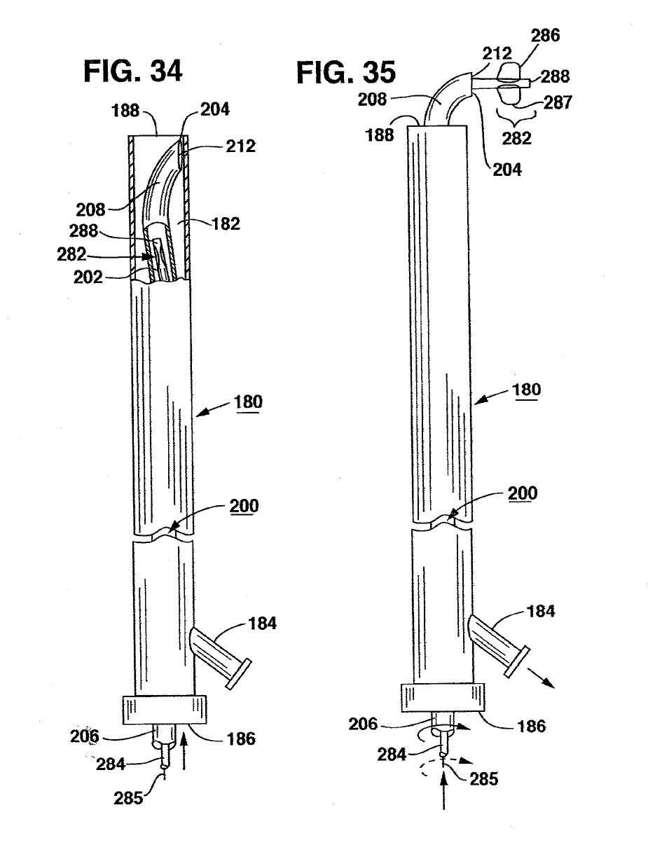

In a further variation depicted in FIGS. 34-36 that is similar to the discectomy instrument 110, the discectomy instrument 280 comprises a discectomy instrument shaft 284 formed with a shaft lumen extending between the discectomy instrument shaft open proximal end to a closed shaft distal end 288. One or more elongated slit extending parallel to the shaft axis is formed from the shaft lumen through the discectomy instrument shaft wall. An extension or push wire 285 within the discectomy instrument shaft lumen is attached at its distal end to cutting wires 286 and 287 that extend to the shaft distal end 288 where they can be attached or looped back proximally. The cutting wires 286 and 287 are formed of spring wire that is normally straight when the push wire 285 is slack as shown in FIG. 34. The extension wire 285 within the discectomy instrument shaft lumen can be advanced distally from the proximal end to bow the cutting wires 286 and 287 outward from the side openings or slits through the discectomy instrument shaft adjacent to the shaft distal end 288. The degree of outward bowing can be controlled from the proximal end of the discectomy instrument shaft 284, and the push wire 285 can then be locked with the discectomy instrument shaft 284. Then, the assembly of the discectomy instrument 280 and the deflection catheter 200 are rotated at their proximal end by a drive motor. The centrifugal force causes the bowed out cutting wires 286 and 287 to cut through the NP to form a circular disc cavity as shown in FIG. 14. A portion of the AF can also be excised in this manner if the axial bore diverges toward it to form a DO closer to the AF section of interest than to other areas of the spinal disc.

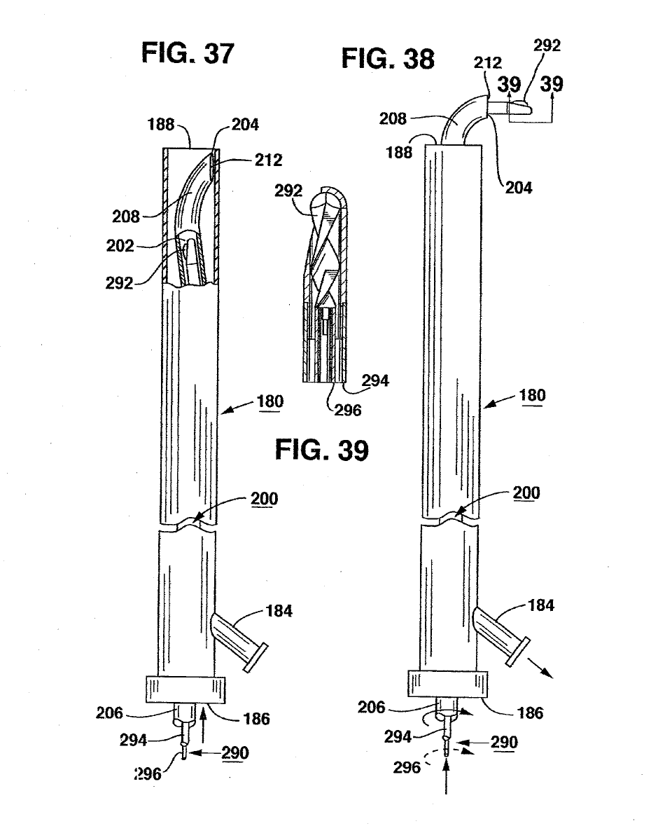

In a still further variation depicted in FIGS. 37-40 that is similar to the discectomy instrument 130, the discectomy instrument 290 comprises a discectomy instrument shaft 294 is formed with a shaft lumen extending between the discectomy instrument shaft open proximal end to a closed shaft distal end 298. A cutting head drive shaft 296 extends through the shaft lumen to a rotatable augur or drill bit 292 that, in this case, is partially shielded by a shaft shield 298, leaving a lateral section of the bit 292 exposed. A drive motor is coupled to the drive shaft proximal end to rotate the cutting head drive shaft 296 and bit 292. Then, the assembly of the discectomy instrument 290 and the deflection catheter 200 are rotated at their proximal end in a prescribed arc or through 360° to form a disc cavity or disc space of the types exemplified in FIGS. 14-16.

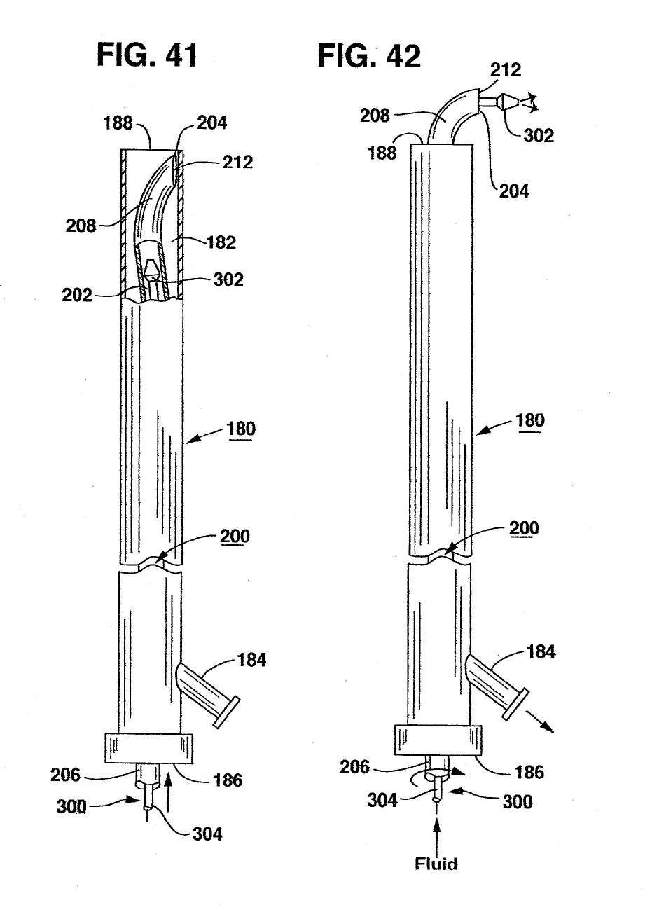

FIGS. 41-43 illustrate yet another type of discectomy instrument 300 of the first type that comprises a fluid jet cutting head 302 that is deflected by the deflection catheter 200 to aim a high pressure fluid jet at a particular direction from the axial disc opening. The fluid jet discectomy instrument shaft 304 is formed with a fluid lumen for conducting pressurized fluid from the discectomy instrument shaft proximal end and out of an axial end port and/or a plurality of side ports to provide an axial distal fluid jet and/or radial fluid jets. The discectomy instrument shaft 304 can be advanced and rotated from the proximal end to locate the fluid jet(s) within prescribed areas of the nucleus and annulus to lyse it into fragments. The emitted fluid and lysed fragments are preferably aspirated through the discectomy sheath lumen that the discectomy instrument shaft 304 is advanced through.

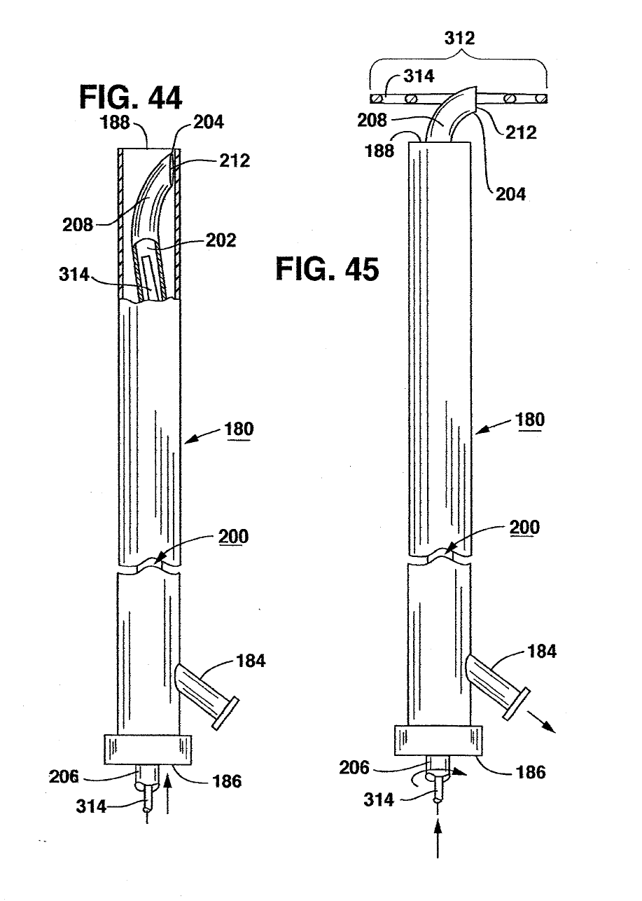

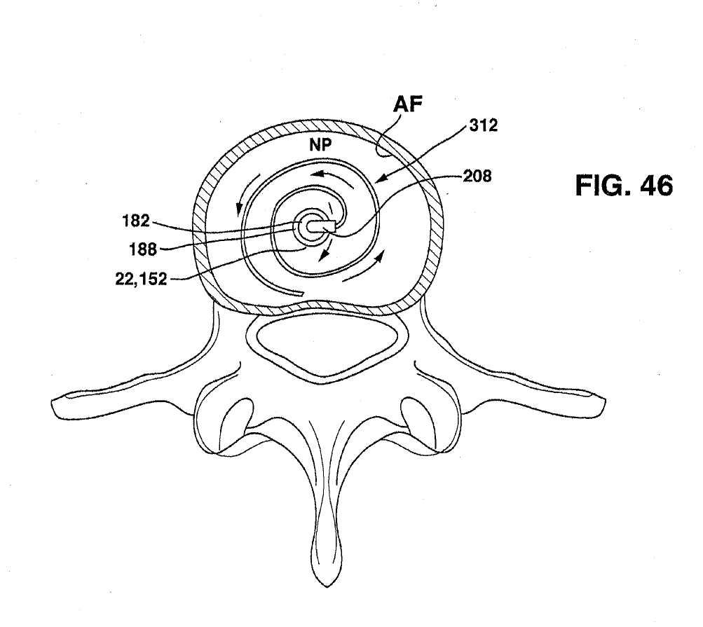

FIGS. 44-46 depict a still further discectomy instrument 310 usable with the deflection catheter 200 as shown or the discectomy sheath 180 alone or directly through the axial bore 22, 152. The discectomy instrument 310 comprises an elongated, flexible, desiccating wire 314 that assumes a planar spiral shape 312 when unrestrained as shown in FIGS. 45 and 46 but is capable of being straightened when inserted through the discectomy sheath lumen or the deflection catheter lumen as shown in FIG. 44. The desiccating wire 314 assumes the planar spiral shape 312 with spiral turns pressing outward against the NP and toward the AF as the desiccating wire 314 is extended out of the distal lumen end opening 212. The proximal end of the deflection catheter 200 can be rotated as the spiral shape 312 is formed, and the outward expansion force of the spiral shape 312 causes the nucleus to desiccate as by compression and separation as the spiral shape 312 forms.

In addition, the proximal portion of the desiccating wire 314 can be insulated from the disc opening to the proximal end thereof by an electrically insulating outer sheath. Electrocautery energy can be applied through the exposed desiccating wire 314 in a unipolar mode to the NP or AF as the wire 314 expands outward and forms the spiral shape 312.

It should be noted that the resistance heating element of the embodiment depicted in FIGS. 22-24 that is energized to delivery thermal energy can be formed to assume the planar spiral shape 312 so that the desiccating energy includes both the outward pressure of the expanding turns as well as the thermal energy conducted to the tissue.

Second Discectomy Instrument Type:

A second preferred type of discectomy instrument comprises a cutting head that extends laterally at about 90° or less outward from the discectomy instrument shaft when the cutting head is released but can be confined within the discectomy sheath lumen or TASIF axial bore during introduction through the TASIF axial bore. These cutting tools of the second preferred type can be rotated by a drive motor to form a constant diameter disc cavity of the type shown in FIG. 14 that is either contained within the annulus or intrudes into a segment of the disc annulus, depending upon the location of the axial disc opening and the extended radius of the cutting head when fully released into the nucleus.

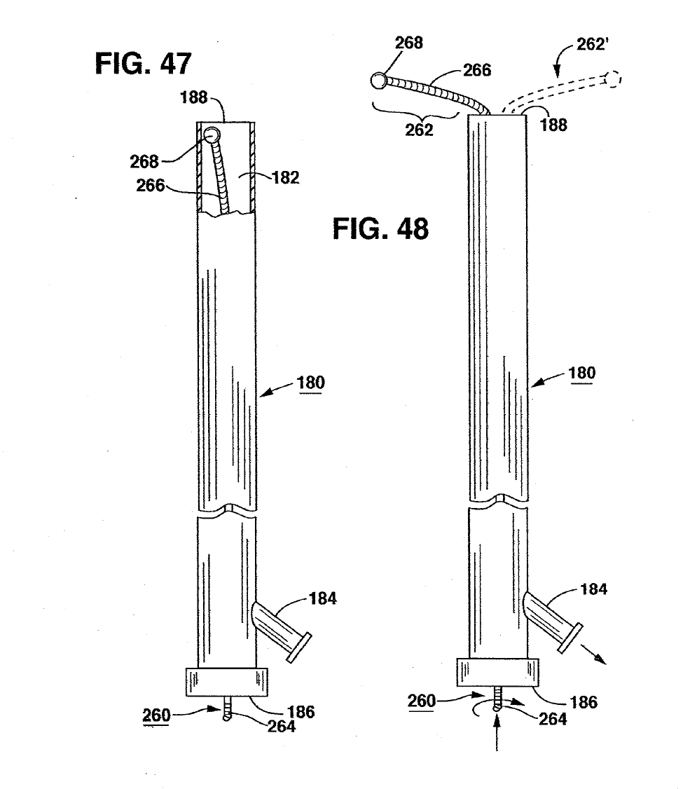

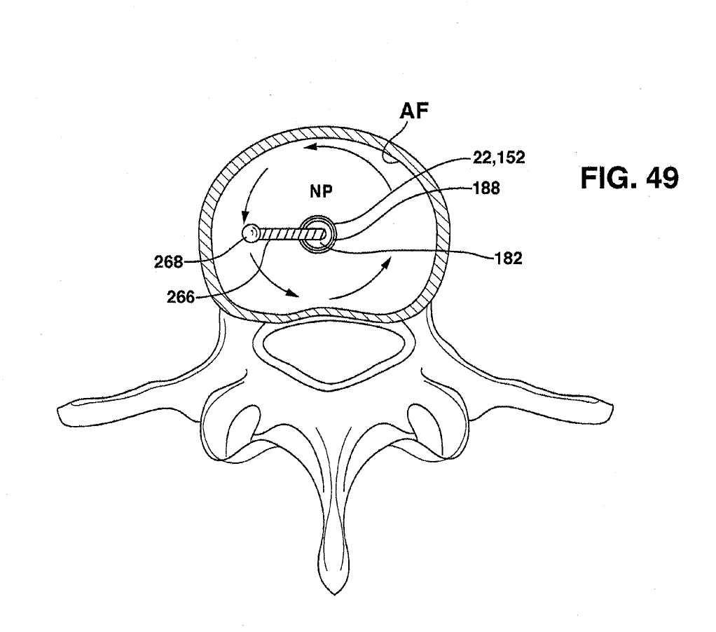

FIGS. 47-49 illustrate that the discectomy instrument 260 described above with respect to FIGS. 28-30 can be employed directly through the discectomy sheath 180. The cutting head 262 is extended laterally as the assembly of the discectomy instrument 260 and the deflection catheter 200 are rotated at their proximal end by drive motor. The centrifugal force causes the cutting head 262 to cut through the NP to form a circular disc cavity as shown in FIG. 14. A portion of the AF can also be excised in this manner if the axial bore diverges toward it to form a DO closer to the AF section of interest than to other areas of the spinal disc. A first cutting head 262 and a second (or more) cutting head 262′ shown in broken lines can be formed as branches of the shaft 264.

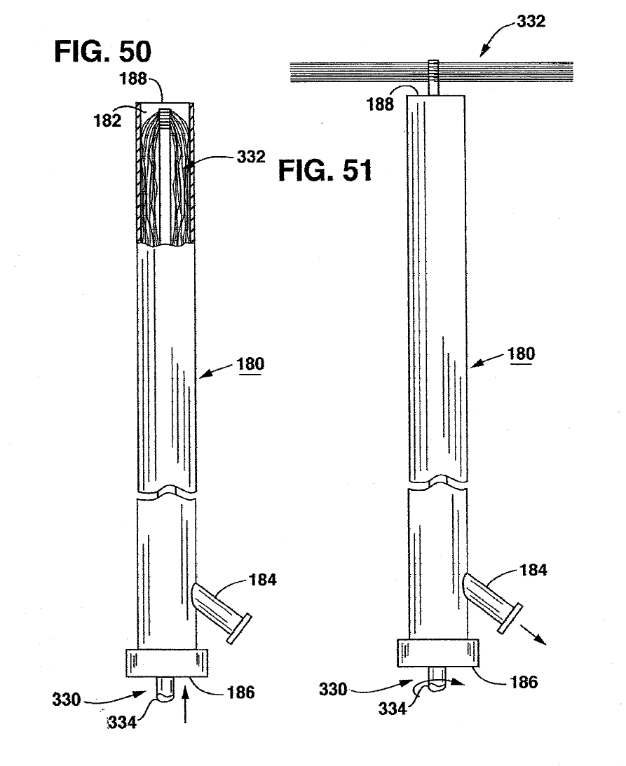

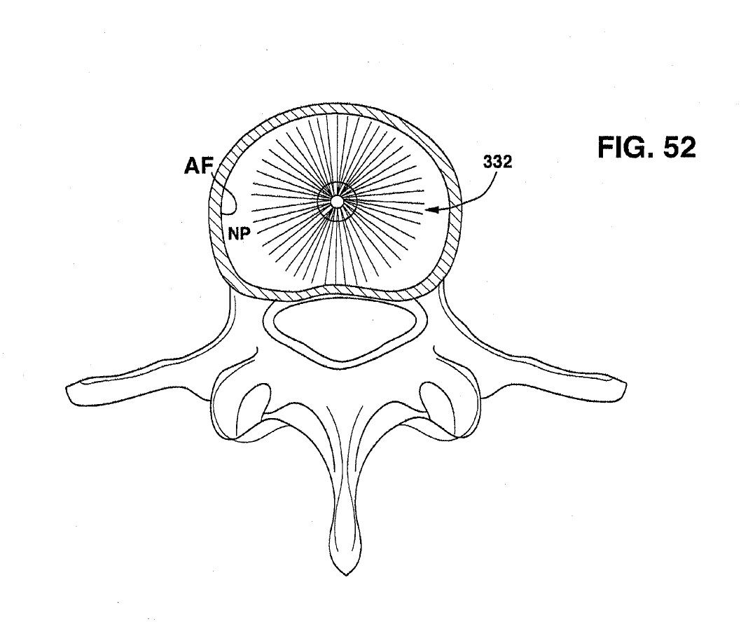

FIGS. 50-52 depict a further discectomy instrument 330 comprising an elongated drive shaft 334 with a distal cutting head brush 332 comprising brush filaments that are stiff enough to extend laterally when unrestrained as shown in FIGS. 51-52 but can be bent against the drive shaft 334 when inserted into the discectomy sheath lumen as shown in FIG. 50. The length of the brush filaments and the height of the brush 332 can be selected to optimally pass through the disc opening and enter the nucleus. The disc filaments are deployed in the nucleus by rotation of the drive shaft. It may be desirable in this instance to have the brush filaments extending proximally in the sheath lumen as shown in FIG. 50 or distally in the sheath lumen before the brush 332 is advanced through the axial disc opening. It may also be desirable to form the axial bore to extend in the cephalad direction into the cephalad vertebral body to enable back and forth manipulation of the drive shaft 334 to locate the filaments within the disc nucleus. Once deployed, the brush filaments are rotated to brush apart the nucleus and form a disc cavity or disc opening.

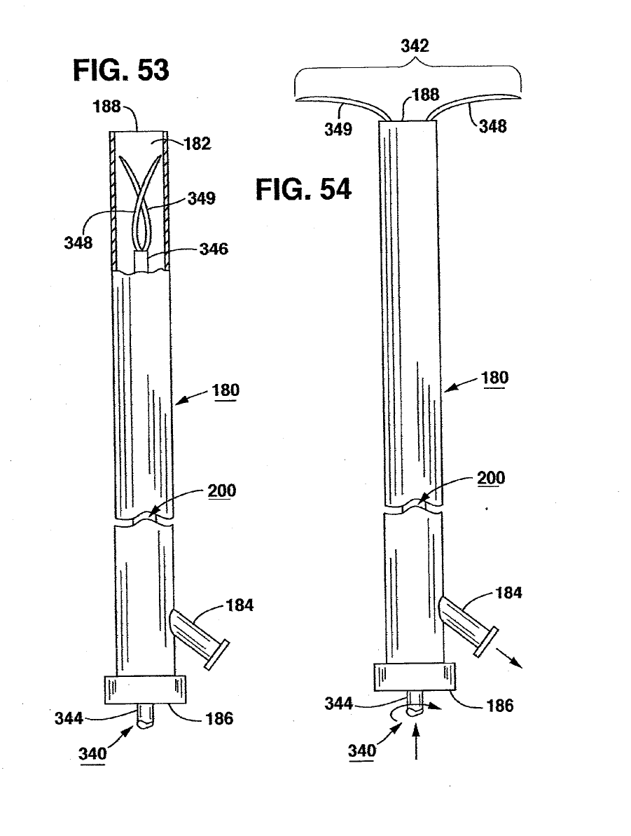

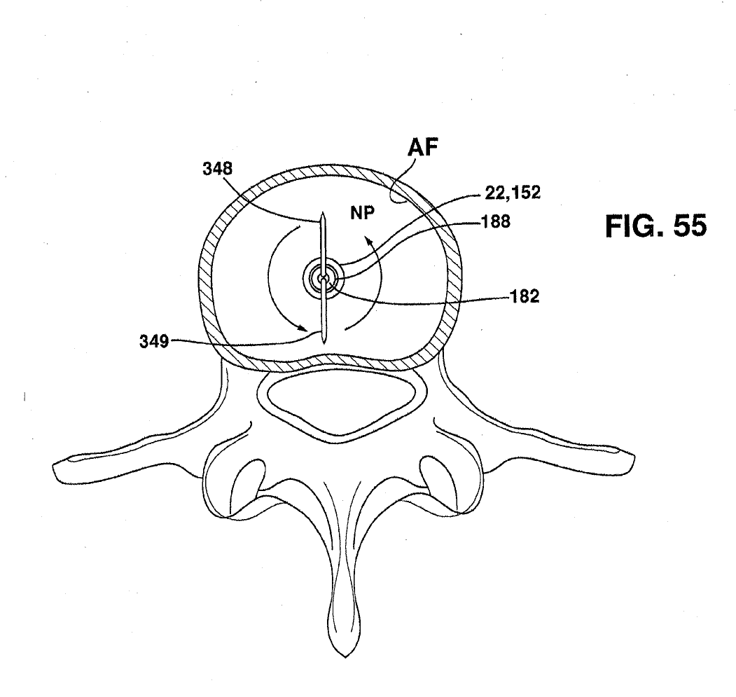

FIGS. 53-55 depict a further discectomy instrument 340 comprising an elongated drive shaft 344 with a distal cutting head 342 comprising a pair of blades 348 and 349 attached to the shaft distal end 346 and that extend laterally when unrestrained as shown in FIGS. 54-55 but can be bent or folded against the discectomy sheath wall when restrained in the discectomy sheath lumen as shown in FIG. 53. The lengths of the blades 348 and 349 can be selected to optimally pass through the disc opening and enter the nucleus. It may also be desirable in this instance to have the blades 348 and 349 extending distally in the sheath lumen as shown in FIG. 53 or proximally in the sheath lumen before they are advanced through the axial disc opening. It may also be desirable to form the axial bore to extend in the cephalad direction into the cephalad vertebral body to enable back and forth manipulation of the drive shaft 344 to locate the blades 348 and 349 within the disc nucleus. Once deployed, the blades 348 and 349 are rotated to cut apart the nucleus and form a disc cavity or disc opening.

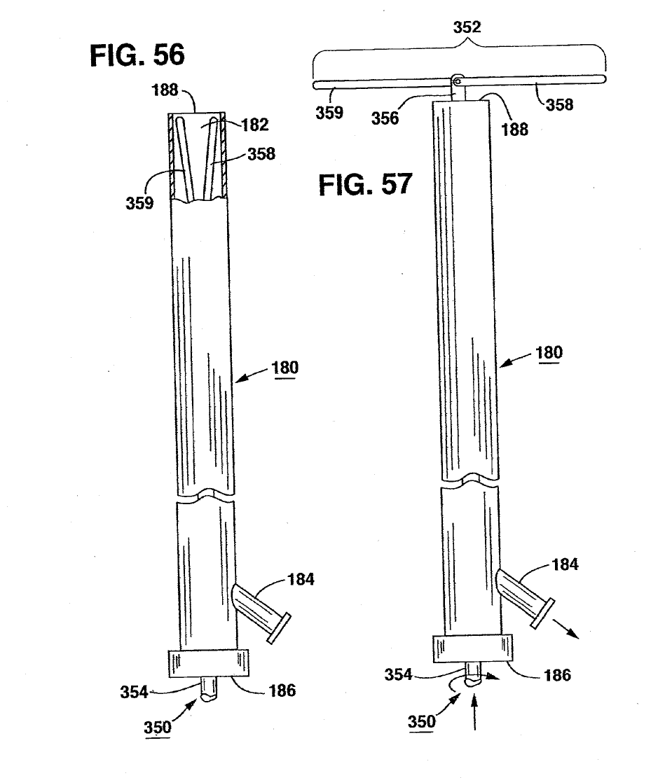

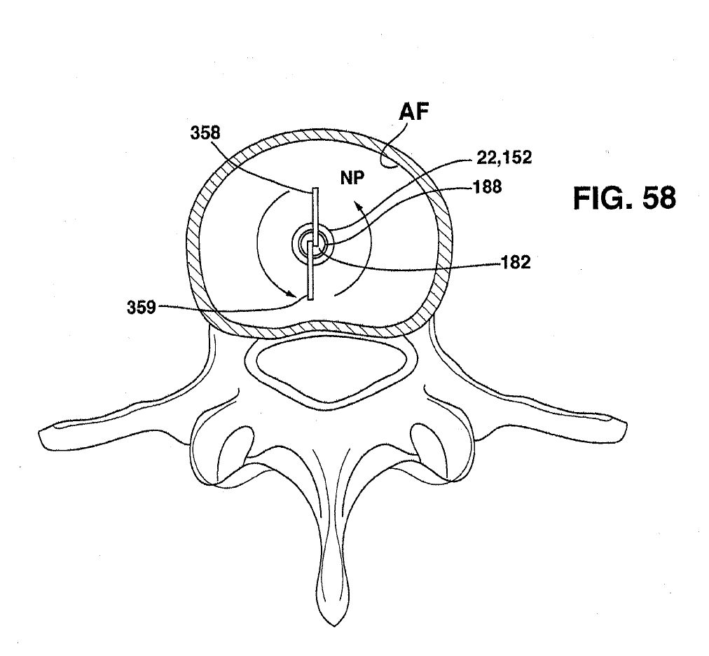

FIGS. 56-58 depict a still further discectomy instrument 350 comprising an elongated drive shaft 354 with a distal cutting head 352 comprising a pair of paddles 358 and 359 hinged to the shaft distal end 356 and that extend laterally when unrestrained and rotated as shown in FIGS. 57-58 but can be folded against the discectomy sheath wall when restrained in the discectomy sheath lumen as shown in FIG. 56. The lengths and lengthwise flexibility of paddles 358 and 359 can be selected to optimally pass through the disc opening and enter the nucleus. It may also be desirable in this instance to have the paddles 358 and 359 extending proximally in the sheath lumen as shown in FIG. 56 or distally in the sheath lumen before they are advanced through the axial disc opening. It may also be desirable to form the axial bore to extend in the cephalad direction into the cephalad vertebral body to enable back and forth manipulation of the drive shaft 354 to locate the paddles 358 and 359 within the disc nucleus. Once deployed, the paddles 358 and 359 are rotated to cut apart the nucleus and form a disc cavity or disc opening.

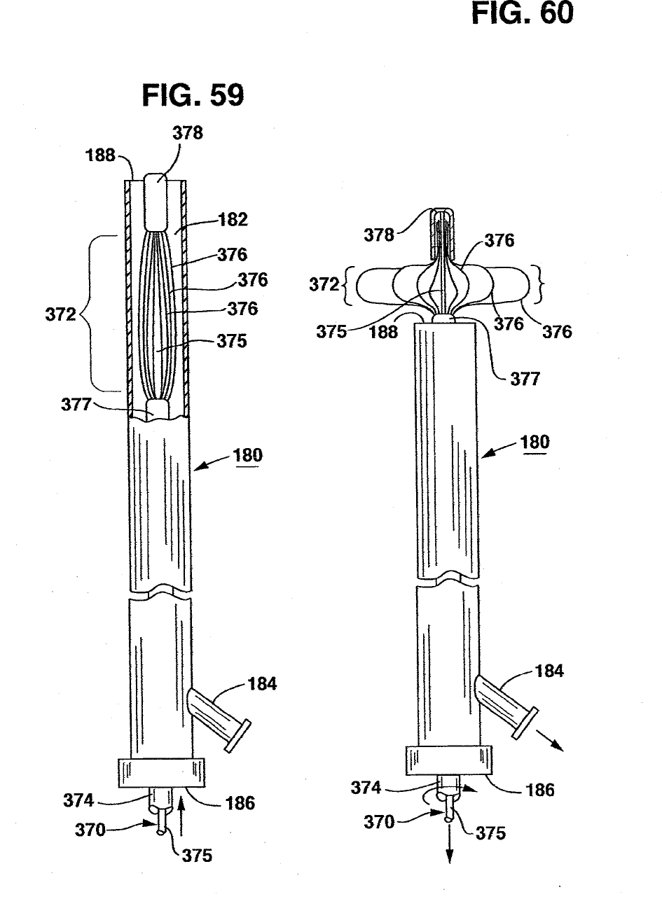

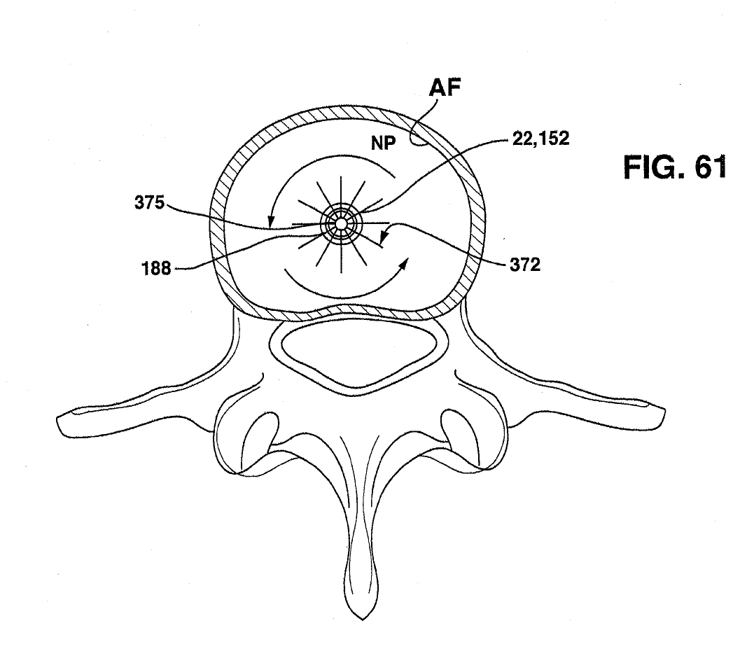

FIGS. 59-61 depict a still further discectomy instrument 370 comprising an elongated drive shaft 364 supporting a distally extending cutting head 372 formed of a plurality of cutting wires 376 extending from the shaft distal end 377 to a common connection at a cutting head distal end 378. The discectomy instrument shaft 374 is formed with a shaft lumen extending between the discectomy instrument shaft proximal and distal ends. A retraction or pull wire 375 within the discectomy instrument shaft lumen is attached at its distal end to the cutting head distal end 378. The cutting wires 376 are formed of spring wire that is normally straight when the pull wire 375 is slack as shown in FIG. 59 but that can be bowed outward as the cutting head distal end 378 is retracted proximally when the pull wire 375 is retracted as shown in FIGS. 60 and 61.

In this case, the TASIF axial bore 22, 152 extends into the cephalad vertebral body providing caudal and cephalad axial disc openings. The cutting head 372 is advanced distally past the sheath distal end 188 through both disc openings while in the relaxed state depicted in FIG. 59. Then, the pull wire 375 is retracted and the axial location of the bowed out cutting wires is adjusted by back and forth movement of the shaft 374 to center the outwardly bowed cutting wires in the disc nucleus while the movements are under visualization. The degree of outward bowing can be controlled from the proximal end of the discectomy instrument shaft 374, and the pull wire 375 can then be locked with the discectomy instrument shaft 374. Then, the discectomy instrument shaft 374 and the pull wire 375 are rotated at their proximal end by drive motor. The centrifugal force causes the bowed out cutting wires 376 to cut through the NP to form a circular disc cavity as shown in FIG. 14. A portion of the AF can also be excised in this manner if the axial bore diverges toward it to form a DO closer to the AF section of interest than to other areas of the spinal disc.

### SUMMARY

The above described TASIF axial bores from the anterior or posterior sacral positions to the axial disc openings are preferably filled, plugged or closed following the discectomy with a plug or bone growth material or bone cement. It will also be understood that the discectomy procedure can be conducted on more than one spinal disc accessed or traversed by at least one TASIF axial bore. For example, two intervertebral spinal discs may be accessed by a single TASIF axial bore, and a discectomy performed in one of the following ways, starting with the cephalad spinal disc. Then, the portion of the TASIF axial bore between the cephalad and caudal spinal disc is closed by an artificial axial spinal implant or bone growth material as appropriate. A discectomy of the caudal spinal disc is then performed, and the portion of the TASIF axial bore between the caudal spinal disc and the anterior or posterior sacral bore entry point is closed by an artificial axial spinal implant or bone growth material as appropriate. Similarly, cephalad and caudal vertebral bodies may be treated by vertebroplasty or balloon-assisted vertebroplasty, and the intervertebral disc may also be treated by one of the following described therapies. For convenience, the treatment of only a single spinal disc or vertebral body is described and illustrated in the drawings.

For purposes of achieving fusion or filling a TASIF axial bore, a “bone growth material” can be one or more of the following, or any other biocompatible material judged to have the desired physiologic response, including any natural or artificial osteoconductive, osteoinductive, osteogenic, or other fusion encouraging material. Particularly, morselized cortical, cancellous, or cortico-cancellous bone graft, including autograft, allograft, or xenograft might be employed. Or any bone graft substitute or combination of bone graft substitutes, or combinations of bone graft and bone graft substitutes, or bone inducing substances, could be employed. Such bone graft substitutes or bone inducing substances include, but not limited to, hydroxyapatite, hydroxyapatite tricalcium phosphate; bone morphogenic protein (BMP) and calcified or decalcified bone derivative and resorbable bone cements. The resorbable cement material can be a calcium derivative generally composed of hydroxyapatite, orthophosphoric acid, calcium carbonate, and calcium hydroxide formed into a semi-liquid paste with an alkaline solution of sodium hydroxide and water or a composition comprising polypropylene fumarate or a mixture of calcium phosphates. Other compositions that may be employed comprise calcium salt filler, N-vinyl-2-pyrrolidone, and a peroxide or free radical initiator. The bone graft material may be mixed with a radiographic material to enable its visualization during delivery to assure proper disposition and filling of bores, cavities and spaces described herein.

In all of the above-described procedures, the visualization of the spine and the introduction of instruments employed to form the anterior or posterior axial bore(s) or to perform therapies, and any spinal disc implants or axial spinal implants or other implanted medical devices is effected employing conventional imaging techniques including open MRI, fluoroscopy or ultrasound through the patient's body or using endoscopic techniques through an axial bore.

All patents and other publications identified above are incorporated herein by reference.

While the present invention has been illustrated and described with particularity in terms of preferred embodiments, it should be understood that no limitation of the scope of the invention is intended thereby. The scope of the invention is defined only by the claims appended hereto. It should also be understood that variations of the particular embodiments described herein incorporating the principles of the present invention will occur to those of ordinary skill in the art and yet be within the scope of the appended claims.

What is claimed is:

1. An assembly for cutting material inside an intervertebral spinal disc, the intervertebral spinal disc having a disc body formed of a nucleus and annulus, through a trans-sacral axial bore extending cephalad and axially from a sacral position of a sacral vertebral body through one or more vertebral body and through a vertebral body endplate and axial disc opening into the nucleus of the intervertebral spinal disc, the assembly comprising:
an elongated discectomy instrument having a discectomy instrument body extending between a discectomy instrument proximal end and instrument distal end, a cutting head located in a distal portion of the discectomy instrument, the instrument body and cutting head dimensioned to fit within and to extend through the axial bore;
a sheath, coaxially carried by the discectomy instrument and within which the discectomy instrument is axially moveable, the cutting head movable between a laterally extending orientation when unconstrained by the sheath, and an axial orientation when constrained by the sheath; and
an anterior tract sheath having a distal end for engaging an anterior surface of the sacral vertebral body.

2. The apparatus of claim 1, further comprising aspiration means for aspirating a disc cavity or disc space within the intervertebral spinal disc.

3. The apparatus of claim 1, wherein the cutting head comprises a fragmenting element for fragmenting the nucleus or annulus into fragments to form a disc cavity or disc space within the intervertebral spinal disc.

4. The apparatus of claim 3, further comprising aspiration means for aspirating nucleus or annulus fragments from the disc cavity or disc space.

5. The apparatus of claim 3, further comprising:
irrigation means for delivering irrigation fluid into the disc cavity or disc space; and
aspiration means for aspirating the nucleus fragments and irrigation fluid from the disc cavity or disc space.

6. The apparatus of claim 1, further comprising:
means for accessing the sacral position of the sacral vertebral body; and
means operable from the accessed sacral position for boring a trans-sacral axial bore cephalad and axially through a series of adjacent vertebral bodies and any intervening spinal discs.

7. The apparatus of claim 1, further comprising:
means for accessing the anterior surface of the sacral vertebral body; and
means operable from the accessed anterior surface for boring a trans-sacral axial bore cephalad and axially through a series of adjacent vertebral bodies and any intervening spinal discs.
